(19) United States
(12) Patent Application Publication
(10) Pub. No.: US 2024/0229155 A1
(43) Pub. Date: Jul. 11, 2024

(54) METHYLATION MARKERS FOR THE TREATMENT OF GLIOBLASTOMA AND OTHER CANCERS

(71) Applicant: Duke University, Durham, NC (US)

(72) Inventors: David ASHLEY, Durham, NC (US); Justin LOW, Durham, NC (US); Michelle BOWIE, Durham, NC (US)

(21) Appl. No.: 18/548,025

(22) PCT Filed: Feb. 25, 2022
(86) PCT No.: PCT/US2022/017985

§ 371 Date: Mar. 18, 2024

Related U.S. Application Data
(60) Provisional application No. 63/307,419, filed on Feb. 7, 2022.
(60) Provisional application No. 63/153,450, filed on Feb. 25, 2021.

(51) Int. Cl. C12Q 1/6886

(52) U.S. Cl. CPC C12Q 1/6886; C12Q 2600/106; C12Q 2600/136; C12Q 2600/154

(57) ABSTRACT

The present disclosure describes methylation markers in the STING promoter useful for making treatment decisions regarding certain cancer and tumors, such as cranial cancers or extracranial cancers of neuroectodermal embryologic origin. The present disclosure also describes methylation markers in the STING promoter for making treatment decisions regarding brain malignancies. These methylation markers serve as indicators of the likelihood of the tumor or cancer being responsive to certain therapeutic modalities, such as an immunotherapy. The present disclosure also describes methods of decreasing the methylation state of the STING promoter in a patient via the administration of a demethylating agent to improve outcomes and increase the likelihood of responding positively to therapies.

### CROSS-REFERENCE TO RELATED APPLICATIONS

This patent application claims the benefit of priority of U.S. Provisional Patent Application No. 63/153,450, filed Feb. 25, 2021 and U.S. Provisional Patent Application No. 63/307,419, filed Feb. 7, 2022, both of which are incorporated herein by reference in their entirety.

### SEQUENCE LISTING

This application is being filed electronically via EFS-Web and includes an electronically submitted Sequence Listing in .txt format. The .txt file contains a sequence listing entitled “155554.00636_ST25.txt” created on Feb. 25, 2022 and is 5,132 bytes in size. The Sequence Listing contained in this .txt file is part of the specification and is hereby incorporated by reference herein in its entirety.

### BACKGROUND

Stimulator of interferon genes (STING) is an important component of the innate immune response to pathogenic DNA. STING senses the presence of DNA in the cytoplasm, an environment that is normally DNA-free. STING is activated by cytosolic cyclic dinucleotides, which may be released by bacteria or created through the interaction of cytosolic self-DNA with cyclic GMP-AMP synthase (cGAS). Activated STING undergoes a conformational change that results in its association with TANK-binding kinase 1 (TBK1), translocation to perinuclear regions, and binding to the transcription factor IRF3. Upon phosphorylation by TBK1, activated IRF3 translocates to the nucleus where it promotes the transcription of target cytokines and type I interferons. Initially described in response to viral and bacterial infection, the importance of STING in stimulating innate immune responses has been recently recognized in multiple cancer types, including mouse glioma models. STING signaling is disrupted in colorectal cancer and melanoma. A pan-cancer analysis showed that STING signaling is commonly disrupted across cancer through loss-of-function mutations or hypermethylation of the STING or cGAS promoters. As STING function and expression is disrupted across various cancers via methylation of the STING promoter, there is a need in the art for methods of monitoring the methylation status of the STING promoter in tumor cells of patients to inform treatment decisions. Further, for patients identified as having unfavorable STING promoter methylation, there is a need in the art for methods that increase STING activity to promote better treatment outcomes, such as for therapeutic modalities aided by increased or normalization of STING expression and function.

### SUMMARY OF THE DISCLOSURE

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, the present disclosure provides a method of treating a cancer in a subject. The method includes obtaining a sample from the subject comprising a tumor cell and determining a methylation status of at least one CpG in a TMEM173 (STING) promoter in the sample. If the sample is determined to have one or more CpG methylated or is hypermethylated at the STING promoter, then a demethylating agent is administered to the subject. The cancer may be a cranial cancer or an extracranial cancer of neural crest or neuroectodermal embryologic origin.

In another aspect, the present disclosure provides a method of predicting the responsiveness of a cancer in a subject to an immunotherapy. The method includes obtaining a sample from the subject comprising a tumor cell and determining a methylation status of at least one CpG in a TMEM173 (STING) promoter in the sample. If the sample is determined to have one or more CpG unmethylated or is hypomethylated, then an immunotherapy is administered to the subject. The cancer can be a cranial cancer or an extracranial cancer of neural crest or neuroectodermal embryologic origin.

In yet another aspect, the present disclosure provides a method of treating a cancer in a subject. The method includes administering a therapeutically effective amount of a demethylating agent to a subject in need thereof. The cancer can be a cranial cancer or an extracranial cancer of neural crest or neuroectodermal embryologic origin. In some embodiments, the method further includes obtaining a sample from the subject comprising a tumor cell, and detecting methylation of a TMEM173 (STING) promoter in the sample prior to administering the demethylating agent and/or after administration of the demethylating agent to determine a methylation status of the TMEM173 (STING) promoter before and/or after the administration.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which

FIGS. 1A-1B. (A): STING (TMEM173) gene methylation beta values for paired samples from 21 glioblastoma (GBM) patients. Rows represent individual samples. Columns represent CpG sites within the Illumina 450K array and are organized by position in the gene. TSS 200 and 1500 denote sites 200 and 1,500 nucleotides upstream of the transcriptional start site, respectively. Promoter-associated CpG sites are labelled in green. Boxes are color-coded by beta value, with red representing higher and blue lower methylation values. The cg16983159 site that anticorrelates most strongly with RNA expression in TCGA is outlined in black. (B): RNA expression versus STING cg16983159 methylation from TCGA GBM data.

FIG. 2: STING gene methylation beta values across cancer using the same labelling and color scheme as FIG. 1. Each box represents the average methylation value for each indicated dataset (rows) and CpG site (columns). The present GBM dataset is displayed as the first row. TCGA abbreviations are used for TCGA data. For non-TCGA datasets, the GEO accession number is provided. From top to bottom data are organized by primary brain tumors, systemic tumors of neuroectodermal origin, other systemic tumors, and brain metastases.

FIGS. 3A-3B: Tumor purity correlations with STING cg16983159 methylation for the (A) present dataset and (B) TCGA GBM data using calculations from Aran D et al 2015.

FIG. 4: Single-cell RNA expression data for a glioblastoma patient showing co-expression of STING (TMEM173) with immune (PTPRC) and vascular smooth muscle (COL3A1) but not tumor (SOX2) markers.

FIGS. 5A-5C. STING methylation correlates with expression in GBM cell lines. (A) STING promoter cg16983159 methylation for 8 cell lines as reported by CCLE (white circles) and measured by our qPCR-based site-specific methylation assay (grey triangles) and Illumina Infinium EPIC array (black circles). Two biologicals were performed for each cell line. (B) qPCR measuring STING mRNA expression for the same cell lines. RNA was run in biological duplicate, and expression is normalized to beta actin. (C) Western blots showing STING protein expression. Images are representative of duplicate biological repeats.

FIGS. 6A-6B. Dysregulation of the cGAS/STING innate sensing pathway in GBM cell lines. (A) Baseline protein expression of key innate signaling proteins in the cGAS/STING pathway. Western images are representative of duplicate biological repeats. (B) Response to cGAS (ISD) and STING (cGAMP) agonists. Cells were treated with 1.0 mg/mL ISD or ISD control, 10 g/mL 2′3′-cGAMP, 1.0 mg/mL self-gDNA, or liposomal reagent for 4 hours (h) and 24 h. Protein harvested at 4 h and 24 h after agonist treatment. Treatments were done at least in duplicate. Upper panel shows activation of immediate downstream effectors pTBK1 and pIRF3. LN18 and M059J express both baseline STING and cGAS and stimulate both pTBK1 and pIRF3. Lower panel shows makers of interferon response. Only M059J activates all markers in response to all agonists tested.

FIGS. 7A-7C: Decitabine induces cGAS and STING expression and restores STING signaling. (A) Percent methylation at STING promoter CpG site cg16983159 (custom site-specific qPCR-based assay) and STING mRNA expression (qPCR) following 250 nM Decitabine pretreatment or DMSO control for 5 days. Methylation and expression are normalized relative to untreated. Beta actin is used to normalize expression. Mean+/−SD=2 independent experiments. * p-value <0.01, ** p-value <0.05, unpaired t-test. (B) Western blot analysis showing DNMT1, cGAS, and STING expression following Decitabine or DMSO treatment. (C) Western blot analysis for STING pathway activation. Cells were treated with 250 nM Decitabine for 5 days, followed by addition of 10 mg/mL 2′3′-cGAMP. Protein was harvested at 4 h and 24 h post cGAMP treatment.

FIG. 8: Box-and-whisker plots of immune scores for cancers with average STING cg16983159 methylation values >0.6 (black) and <0.3 (grey).

FIGS. 9A-9B: Human Protein Atlas expression data for (A) cGAS and STING in human glioma samples, and (B) STING in normal human tissues. Top panel STING mRNA expression (www.proteinatlas.org/ENSG00000184584-TMEM173/summary/rna), bottom panel STING protein expression (www.proteinatlas.org/ENSG00000184584-TMEM173/tissue).

FIG. 10: Summary of correlations between methylation and mRNA expression for each STING Illumina 450K CpG site tested in the TCGA GBM dataset.

FIGS. 11A-11C: STING expression in developing human cortex cells. (A): t-SNE (t stochastic neighbor embedding) plot showing 4,300 cells from the developing human brain collected 6 to 37 weeks post conception, colored by cell type as determined by single cell RNA-sequencing analysis. (B): t-SNE colored by STING (TMEM173) RNA expression level. (C): Violin plot showing relative expression level in developing cortex cell types. TPM expression is shown. Single cell RNA-seq data are from Nowakowski et al., 2017, Science. Abbreviations: IN-CTX-CGE, central ganglionic eminence-derived inhibitory neurons; IPC-div1/2, dividing intermediate radial glia-like progenitor cells; MGE-div, dividing MGE progenitors; RG-div1, dividing radial gliaG2/M-phase; RG-div2, dividing radial glia (S-phase); EN-PFC, early and late born excitatory neuron prefrontal cortex; EN-V1-2, early born deep layer excitatory neuron V1-2; RG-early, early radial glia; Glyc, glycolysis; IPC-nEN1/2/3, non-dividing intermediate progenitor cells; nIN1/2/3/4/5, medial ganglionic eminence neurons; MGE-IPC1/2/3, medial ganglioniceminence progenitors; MGE-RG1/2, MGE radial glia; IN-CTX-MGE1/2, MGE-derived inhibitory neuron; nEN-earlyl/2, newborn excitatory neuron; nEN-late, late-born excitatory neuron; OPC, oligodendrocyte precursor cell; oRG, outer radial glia; IN-STR, striatal neurons; tRG, truncated radial glia; U1/2/3/4, unknown cell type; and vRG, ventricular radial glia.

FIG. 12: Individual GBM patient cg16983159 methylation b-values before and after treatment.

FIGS. 13A-13F: (A): Fresh glioma tumor tissue was digested to single cell suspensions and CD14 positive selection was performed. Cell count equalized pre-depleted and CD14-depleted samples were compared to the CD14+ enriched fraction for cytokine secretion after 2′3′-cGAMP treatment (48 h). P-values are from Man-Whitney test (two-tailed); boxes indicate median and quartiles, whiskers indicate range; values were normalized to mock treated, pre-depleted samples. After ex vivo slice culture treatment, slices were dissociated to single cell suspensions and tested for IFIT1 expression in tumor-associated myeloid cells (CD14+, CD11b+) vs tumor cells (CD45, CD31−) by flow cytometry (n=3 tumors; p value is from Man-Whitney test, two-tailed). (B) Single-cell RNA expression data for three newly diagnosed GBM patients showing expression of STING (TMEM173) in immune (PTPRC) and vascular smooth muscle/stroma (COL3A1) but not tumor (SOX2) cells. (C) Multiplex immunofluorescence (mIF) analysis of STING, immune, stroma, and tumor marker co-expression in GBM tumor sections. Tissue sections from a GBM patient were analyzed by mIF staining for the expression of CD64 (macrophage, cyan), CD3 (T cells, white), CD31 (endothelial cells, green), vimentin (tumor cells and mesenchymal cells, magenta), and STING (orange). DAPI (blue) was used as the nuclear counterstain. (D): Average STING (TMEM173) gene methylation beta values for each Illumina CpG site (rows) for normal brain controls from fetal [(Price et al., 2016), (Chatterton et al., 2017)] and adult (Gasparoni et al., 2018) tissues and for 64 GBM patient samples, including 40 samples from our previously described cohort (Muscat et al., 2018) and 24 additional new samples. TSS 200 denotes sites between the transcriptional start site (TSS) and 200 nucleotides upstream; and TSS 1500 denotes sites between 200 and 1,500 nucleotides upstream of the TSS. Promoter-associated CpG sites are labelled in blue; the minimal promoter extends up to 124 nucleotides (nt) upstream of the TSS (Wang et al., 2016). Boxes are color-coded by beta value, with red representing higher, yellow intermediate, and green lower methylation values. Pearson's correlation coefficients (r) between methylation beta values and RNA expression from TCGA GBM data are shown for the 7 CpG sites for which these data are available (adjusted p values by step-down Bonferroni correction were all <0.05). The cg16983159 site that anticorrelates most strongly with RNA expression in TCGA is outlined in black (n=46; r=−0.76; adjusted p<10-4). Sequencing of bisulfite converted DNA from two GBM cell lines was performed to semi-quantitatively estimate the percent methylation for each CpG site in a ˜400 nt region centered on cg16983159. Cell lines were pre-treated with decitabine (DAC) or DMSO control. (E): Decitabine induces STING expression and restores STING signaling. Top panel: Percent methylation at STING promoter CpG site cg16983159 (site-specific qPCR-based assay) and STING mRNA expression (qPCR) following 250 nM decitabine pretreatment or DMSO control for 5 days. RNA expression is shown relative to untreated controls. Beta actin is used to normalize expression. Results for each of 2 independent experiments per cell line are shown as dots and their averages as grey and white rectangles for methylation and RNA expression, respectively. Middle panel: Western blot analysis showing DNMT1 and STING expression following decitabine or DMSO treatment. Bottom panel: Western blot analysis for STING pathway activation. Cells were treated with 250 nM decitabine for 5 days, followed by addition of 10 μg/mL 2′3′-cGAMP. Protein was harvested at 4 h and 24 h post cGAMP treatment. Images are representative of two experiments; actin serves as loading control. (F): Scatter plot of average cg16983159 beta values for TCGA solid cancers versus their comparison normal tissues. In general, when cancer average beta <0.5, their corresponding normal tissues are also <0.5 (upper right and lower left quadrants). The four exceptions are labelled as follows: red when the cancer displays an average beta >0.5 with normal tissue <0.5 (PRAD: prostate adenocarcinoma); and blue when the cancer displays an average beta <0.5 with normal tissue >0.5 (THCA: thyroid adenocarcinoma, PAAD: pancreatic adenocarcinoma, KIRC: renal clear cell carcinoma). GBM is labelled in green. Breast (BRCA), melanoma (SKCM, UVM), and lung cancer (LUSC, LUAD) are labeled in black. Bar graphs show cg16983159 methylation values for other primary brain tumors (black) and for extracranial neuroectoderm-derived tumors (grey). ATRT: atypical teratoid rhabdoid tumor, LGG: low grade glioma, Medullo: medulloblastoma, DIPG: diffuse intrinsic pontine glioma, PA: pilocytic astrocytoma, Epen: ependymoma, PCNSL: primary central nervous system lymphoma, Men: meningioma, SINET: small intestine neuroendocrine tumor, Pheo: pheochromocytoma, PanNET: pancreatic neuroendocrine tumor, Pit adeno: pituitary adenoma.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The present disclosure provides a method of treating a cancer in a subject. The methods include obtaining a sample comprising a tumor cell from the subject and determining the methylation status of at least one CpG in a TMEM173 (STING) promoter in the sample. The methylation status can then be used to determine the treatment course for the subject to treat the tumor or cancer in the subject and an anti-cancer therapeutic can be administered to the subject. In some embodiments, the methylation status of at least one CpG in SEQ ID NO: 13 is determined. SEQ ID NO: 13 is shown below with the CpG sites shown to be prone to methylation indicated with larger font letters and the key methylation site at cg1698315 pointed out specifically with an arrow and labeled. In some embodiments, an immunotherapy is administered to the subject if one or more CpG in the TMEM173 (STING) promoter corresponding to those shown below in SEQ ID NO: 13 is unmethylated. In some embodiments, an immunotherapy is administered to the subject if more than one or all of the CpG in the TMEM173 (STING) promoter corresponding to those shown below in SEQ ID NO: 13 is unmethylated. In some embodiments, a demethylating REPLACEMENT SHEET agent is administered to the subject if one or more CpG in the TMEM173 (STING) promoter is methylated. In some embodiments, an immunotherapy is administered to the subject after the administration of a demethylating agent to the subject. In some embodiments, the immunotherapy is administered to the subject after the administration of the demethylating agent and after determining at least one CpG in the TMEM173 (STING) promoter is determined to be unmethylated.

(human Sting promoter region)
SEQ ID NO: 13
TCTCCTGCCTCAGCCTCCCAAGTAGCTAAGATTACAAG [symbol] CCCACCACC
A [symbol] CCTGGCTAATTTTTGTTTTTAGTAGAGATGGGGTTTCACCATGTTG
CTCAGGCTGGTCTTGAACTCCTGACCTCAAGTGATCCACCCATCT [symbol] GC
CTCCCAAAG [symbol] CTGGGATTACAGGCATGAGCCACTGTGCCAGGCCTGCA
ATTACTTTTGCTCCTACCTAATATCATCCCCACAAC [symbol] CCTTCTGGGCA
GAAAC [symbol] GCAGGCTCTCTTGGAGAAGTCACAGG [symbol] TGGCCATTTCCT
GCAAAGAG

[symbol]

[symbol]

DNA methylation of the STING gene TMEM173 is associated with silencing of STING expression which is needed for robust and effective immune responses. The Examples demonstrate that DNA methylation within the cg16983159 region (SEQ ID NO: 13-15) of the STING promoter can lead to epigenetic silencing of STING expression in tumor cells and brain cells. STING attenuation is associated with loss of tumor suppression and retarded immune responses. The methylation status of the STING promoter is a biomarker for whether a disease is likely to respond to immunotherapies. The term immunotherapy may be used interchangeably with immunomodulatory therapy and immunostimulatory therapies. Further, an unfavorable methylation status of the STING promoter may be converted into a more favorable status via the administration of a demethylation agent(s) resulting in increased STING expression and/or normalization of immune responses. By reducing methylation of the STING promoter, immunostimulatory therapies become more effective.

The present disclosure is based on examining STING dysregulation in glioblastoma using cell lines, patient-derived tissues, and published gene expression databases. This work showed that: (i) STING expression is suppressed through promoter hypermethylation, (ii) STING is expressed in infiltrating immune cells in the tumor microenvironment rather than tumor cells; and (iii) the STING promoter is also hypermethylated in normal brain and non-cancer cerebral diseases. In a pan-cancer analysis, it was found that the STING promoter is also hypermethylated in other primary brain tumors and many extracranial tumors of neuronal origin. In contrast, in most other systemic tumors the STING promoter displays a wide range of methylation values. These results suggest that STING epigenetic silencing is a central characteristic of glioblastoma and other brain tumors and can be used as a predictor of the presence of such a tumor. Additionally, the inventors hypothesize that STING promoter methylation serves as a predictor of responsiveness to various forms of therapy, including immunotherapies across a broad range of cancers. For example, the methylation status of a STING promoter (e.g. a specific methylation pattern) may be used to predict the immunosuppressed state of a tumor or disease. The STING promoter methylation status (e.g. a specific methylation pattern) may be used to predict response to certain therapies immunotherapeutic approaches or lack of responsiveness to such approaches.

The human TMEM173 gene (also referred to herein simply as “TMEM173”) refers to a gene encoding the protein STING. It will be understood by the skilled worker that variations in TMEM173 exist in nature. For example, R232 is a common allele; however, more than 50% of Americans are not R232/R232. HAQ(R71H-G230A-R293Q) is another common allele. H232 is an uncommon allele observed in humans. A representative TMEM173 gene is National Center Biotechnology Institute (NCBI) Gene ID: 340061. Stimulator of Interferon Response cGAMP Interactor 1 (STING), also recognized in the art as ERIS, MITA, MPYS, SAVI, NET23, and STING-beta, is transmembrane protein pattern recognition receptor that functions as a major regulator of innate immune responses to infections. While the name STING might refer to multiple proteins with related structures and polypeptide sequences from various species, for the purposes of the claims, the term “human STING” refers to the protein represented by the predominant sequence UnitProt A0A2R3XZB7 and NCBI accession AVQ94753; however the exact sequence may vary slightly from individual to individual and due to different isoforms, splicing, polymorphisms and/or mutations. The skilled worker will be able to identify other TMEM173 genes and STING proteins, even if they differ from referenced sequences.

As used herein, the promoter region of the human TMEM173 (STING) gene is a region of about 350 to 1,000 base pairs upstream of the transcriptional start site known to the skilled worker. It will be understood by the skilled worker that slight variations in the promoter sequence may exist in nature. In some embodiments, the STING promoter comprises a region consisting of or consisting essentially of SEQ ID NO: 13-15 and/or having at least 85%, 90%, 93%, 95%, 96%, 97%, 98%, 98.5%, 99%, 99.2%, 99.4%, 99.5% or 99.6% identity thereto. The skilled worker will be able to identify a TMEM173 promoter region, if present, in a given genomic sequence.

As used herein, a “methylation site” is a nucleotide within a nucleic acid, nucleic acid target gene region or gene that is susceptible to methylation either by natural occurring events in vivo or by an event instituted to chemically methylate the nucleotide in vitro.

As used herein, a “methylated nucleotide” or a “methylated nucleotide base” refers to the presence of a methyl moiety on a nucleotide base, where the methyl moiety is not present in a recognized typical nucleotide base. Cytosine does not contain a methyl moiety on its pyrimidine ring, however 5-methylcytosine contains a methyl moiety at position 5 of its pyrimidine ring. In this respect, cytosine is not a methylated nucleotide and 5-methylcytosine is a methylated nucleotide.

As used herein, a “methylated nucleic acid molecule” refers to a nucleic acid molecule that contains one or more methylated nucleotides that is methylated, such as, e.g., a cytosine base.

As used herein “CpG island” refers to a G:C-rich region of genomic DNA containing a greater number of CpG dinucleotides relative to total genomic DNA, as defined in the art. It should be noted that differential methylation of the target genes according to the invention is not limited to CpG islands only, but can be in so-called “shores” or can be lying completely outside a CpG island region, called herein more generally a “CpG region” or “CpG site”. In some embodiments, the CpG island(s) are in a genomic sequence annotated by the Illumina, Inc. CpG locus identifier cg16983159. This CpG is shown at base number 279 of SEQ ID NO: 15. The methylation status of the STING promoter may also be based on the methylation of at least one CpG in the STING promoter selected from the CpG sites corresponding to the nucleotides at positions 39, 51, 144, 157, 233, 251 or 279 of SEQ ID NO: 15. Typically, CpG sites and CpG regions are located within the promoter of one or more genes. In the human genome, CpG islands are typically at least 300 base pairs long and can extend from 300 to 3,000 base pairs or more.

### A. Methods of Treating Cancer in a Subject

The methylation patterns of the STING promoter of cells found in neoplastic tissues may be utilized as a sensitive biomarker to predict therapeutic outcomes. The methylation status, level or pattern of at least one CpG in a TMEM173 (STING) promoter in the sample may be used to predict which therapies are more likely to provide a benefit to a subject (e.g. low methylation correlates with responding to immunotherapy) or are more likely to be unsuccessful (e.g. high methylation correlates with no or low response rates to immunotherapy).

The present disclosure provides a method of treating a cancer in a subject. The method includes obtaining a sample from the subject comprising a tumor cell and determining the methylation status of at least one CpG in a TMEM173 (STING) promoter in the sample. The subject is administered a demethylating agent if one or more CpG is methylated. In some embodiments, the methylation status of at least one CpG including the CpG at cg16983159 is determined. In some embodiments, the demethylating agent is administered to the subject if one or more CpG in one of SEQ ID NO: 13-15 is determined to be methylated. In some embodiments, the demethylating agent comprises azacitidine and/or decitabine. In some embodiments, the demethylating agent results in expression of a TMEM173 (STING) gene that was previously silent due to methylation and/or hypermethylation. STING expression may be monitored using a biological sample taken from the subject after administration of a demethylating agent. STING expression may be monitored at the mRNA or protein level. In some embodiments, the method further comprises administering an immunotherapy to the subject. The cancer may be a cranial cancer or an extracranial cancer of neural crest or neuroectodermal embryologic origin.

In another aspect, the present disclosure provides a method of predicting the responsiveness of a cancer in a subject to an immunotherapy. The method may include obtaining a sample from the subject comprising a tumor cell and determining the methylation status of at least one CpG in a TMEM173 (STING) promoter in the sample. An immunotherapy is administered to the subject if one or more CpG is unmethylated. In some embodiments, the methylation status of at least one CpG of cg16983159 is determined. In some embodiments, an immunotherapy is administered to the subject if one or more CpG in SEQ ID NO: 13-15 is unmethylated. In some embodiments, the immunotherapy comprises at least one of an immune checkpoint inhibitor, brain neoantigen vaccine, immunologic adjuvant, and oncolytic virus. The cancer may be a cranial cancer or an extracranial cancer of neural crest or neuroectodermal embryologic origin.

In yet another aspect, the present disclosure provides a method of treating a cancer in a subject by administering a therapeutically effective amount of a demethylating agent to a subject in need thereof. In some embodiments, the method further comprises obtaining a sample from the subject comprising a tumor cell, and detecting methylation of a TMEM173 (STING) promoter in the sample. The detection of the methylation status of the STING promoter can be performed either before and/or after the administering step, such as to monitor any changes to the methylation in response to the demethylating agent. In some embodiments, the demethylating agent comprises azacitidine and/or decitabine. In some embodiments, the demethylating agent results in expression of a TMEM173 (STING) gene that was previously silent due to methylation and/or hypermethylation. STING expression may be monitored using a biological sample taken from the subject, e.g. after administration of a demethylating agent. STING expression may be monitored at the mRNA or protein level. The cancer may be a cranial cancer or an extracranial cancer of neural crest or neuroectodermal embryologic origin.

As used herein, a demethylating agent refers to any agent (e.g. a compound) that inhibits a DNA methylating enzyme and/or removes existing methylation. Non-limiting examples of demethylating agents include nucleoside analogs and DNA methyltransferase inhibitors (DNMTi's) which are not nucleoside analogs. Non-limiting examples demethylating nucleoside analogs include cytidine analogs, such as 5-azacytidine (azacytidine), 5-azadeoxycytidine (decitabine), procaine. A non-limiting example of a DNMTi that is not a nucleoside analog is an antisense nucleotide capable of decreasing or inhibiting expression of one of more DNA methyltransferases. Both pan demethylating agents and agents having more limited demethylating specificities are suitable for use in a method described herein.

As used herein, a cranial cancer is any tumor or cancer of the head, such as within the skull and including within a cranial bone or in the face. For some embodiments of any method of the disclosure, the cancer being treated is a cranial cancer selected from a brain cancer, glioma, glioblastoma (GBM), medulloblastoma, and pituitary adenoma. For other embodiments of any method of the disclosure, the cancer being treated is an extracranial cancer of neuroectodermal embryologic origin selected from a neuroblastoma, pancreatic neuroendocrine cancer, medulloblastoma, and pheochromocytoma. As used herein, an extracranial cancer is any cancer not of the head. For some embodiments of any method of the disclosure, the cancer being treated is brain cancer or CNS cancer. In some embodiments, the cranial cancer or CNS cancer is not a primary CNS lymphoma (PCNSL) and meningioma.

The method may comprise administering an immunotherapy to the subject. In some further embodiments, the immunotherapy comprises at least one of an immune checkpoint inhibitor, a vaccine (e.g., a cancer vaccine), immunologic adjuvant, and oncolytic virus. In some embodiments, the vaccine is a cancer vaccine such as, e.g., a brain neoantigen vaccine.

The methods provided herein may further comprise administering to the subject at least one additional therapeutic agent, such as in addition to a demethylating agent and/or an immunotherapy.

The methods may further comprise resecting one or more tumors from the subject.

The methods may be utilized to measure, detect, determine, identify, and/or characterize the methylation status/level of a STING promoter in a sample. The methylation status of only a subregion or even a single or two CpG of the STING promoter may be determined. The STING promoter region is a region of about 350 to 1,000 base pairs upstream of the transcriptional start.

In some embodiments, the STING promoter comprises a region consisting of or consisting essentially of SEQ ID NO: 13-15. In some embodiments, the subregion comprises or consists essentially of the first 340, 350, 370, 400, or 450 base pairs upstream of the STING transcriptional start site. In some embodiments, the subregion comprising 340 base pairs upstream of the STING transcriptional start site comprises at least four CpG sites of interest. In some embodiments, the CpGs of interest are selected from one or more CpGs positioned relative to the transcriptional start site at −98, −126, −144, −220, −326, and −338 (with minus indicating upstream) (which correspond to 39, 51, 157, 233, 251 and 279 of SEQ ID NO: 15). In some embodiments, the exact CpG sites and/or methylation patterns differ with the type of cancer being analyzed and/or between individual patients.

In some embodiments, the STING promoter comprises a region consisting of or consisting essentially of one of SEQ ID NO: 13-15 and the CpG sites of interest are selected from one or more CpGs positioned at 39-40, 51-52, 144-145, 157-158, 233-234, 251-252, and 279-280 of SEQ ID NO: 13. The skilled worker will be able to determine the equivalent site to any of these if the regions comprises sequence variation or in the sample obtained from a human subject.

In some embodiments, the cancer comprises a STING-silenced cancer. As used herein, the term “STING-silenced cancer” comprise those tumors where the region of the STING promoter near cg16983159 is hypermethylated. These include most primary brain tumors and many tumors of neuroectodermal or neural crest origin. The term STING-silenced cancer also refers to individual tumors of any cancer type where cg16983159 is hypermethylated.

In some embodiments, the cancer comprises a STING-silenced cancer that is immunologically “cold.” Certain methods described herein may transform or alter an immunologically cold tumor into becoming an immunologically active tumor. As used herein, the term “immunologically cold” tumor refers to a tumor that can be characterized by the low levels of certain immune markers, which correlate with being “non-inflamed” and/or excluded from immunosurveillance, e.g. “immune-excluded tumors,” such as a result of T cell anergy or exhaustion. For example, a glioblastoma may be resistant to immunotherapy but after performing a method of the disclosure, that glioblastoma becomes sensitive to immunotherapy.

Disclosed herein are methods of diagnosing, prognosing, prediction, or predicting a STING-silenced cancer, or for providing an indication for susceptibility to an anti-cancer therapy for such a cancer in a subject. In some instances, the methods comprise utilizing one or more biomarkers described herein. In some instances, a biomarker comprises the CpG at cg16983159.

Methods comprising, consisting of, or consisting essentially of (a) analyzing the methylation pattern of the CpG region near cg16983159 in a biological sample from the subject; and (b) comparing the methylation pattern of the region with the methylation pattern of a control sample in which the presence of a hypermethylation pattern of cg16983159 as compared to the control is indicative of a STING-silenced cancer in the subject; and (c) administering an appropriate anticancer therapy or demethylating agent to the subject depending on the pattern detected are provided.

The sample may be obtained from a subject suspected of having a STING-silenced cancer. The cancer may include brain tumors, systemic tumors of neural-crest origin, and any other cancer that may display STING epigenetic silencing. The methods comprise, consist of, or consist essentially of: (a) processing a biological sample obtained from the subject; (b) detecting the methylation pattern of cg16983159 or another CpG site in the STING promoter; and (c) performing a DNA sequencing analysis to determine the methylation pattern of the cg16983159 or another CpG site in the STING promoter.

Methods for identifying an agent that modulates the methylation pattern of a STING-silenced cancer comprising, consisting of, or consisting essentially of. (a) contacting a sample comprising a hypermethylated STING promoter with the agent and (b) analyzing the methylation pattern of the STING promoter in the sample prior to and after contact with the agent.

Another aspect of the present disclosure provides a method of treating a STING-silenced tumor in a subject comprising, consisting of, or consisting essentially of: (a) determining the methylation pattern of the STING promoter in a subject; (b) prognosticating, diagnosing, or predicting clinical outcome for the individual's tumor based upon the methylation pattern; (c) selecting subjects having a poor clinical outcome; and (d) treating the subject having a poor clinical outcome.

Another aspect of the present disclosure provides a method of identifying, amongst all systemic cancers, tumors that are not STING-silenced and thus more likely to respond to traditional anti-cancer therapies including immunotherapy comprising, consisting of, or consisting essentially of: (a) determining the methylation pattern of the STING promoter in a subject; (b) prognosticating, diagnosing, or predicting clinical outcome for the individual's tumor based upon the methylation pattern; (c) selecting subjects predicted to have a favorable response to traditional-anticancer therapy; and (d) treating the subject predicted to have this more favorable treatment response.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In some embodiments, the biological sample is a biopsy (such as a tumor biopsy). A biological sample may be obtained directly from a subject (e.g., by blood or tissue sampling) or from a third party (e.g., received from an intermediary, such as a healthcare provider or lab technician). In some embodiments, the biological sample is selected from the group consisting of tissues, cells, biopsies, blood, lymph, serum, plasma, urine, saliva, mucus and tears. In certain embodiments, the biological sample comprises a biopsy.

In some instances, the methylation profile/status is generated from a biological sample isolated from a subject. In some embodiments, the biological sample is a biopsy (e.g., a tumor biopsy). In some instances, the biological sample is a tissue sample (e.g., a diseased tissue). In some instances, the biological sample is a tissue biopsy sample. In some instances, the biological sample is a blood sample. In some instances, the biological sample is a cell-free biological sample. In other instances, the biological sample is a circulating tumor DNA sample (e.g. a cell-free biological sample containing circulating tumor DNA).

In some embodiments, STING DNA is obtained from a liquid sample. In some embodiments, the liquid sample comprises blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some embodiments, the biological fluid is blood, a blood derivative or a blood fraction, e.g., serum or plasma. In some embodiments, the sample comprises whole blood, serum, and/or plasma. In some embodiments, the sample comprises urine. In some embodiments, the liquid sample also encompasses a sample that has been manipulated in any way after their procurement, such as by centrifugation, filtration, precipitation, dialysis, chromatography, treatment with reagents, washed, or enriched for certain cell populations.

In some embodiments, a DNA comprising a STING gene or fragment thereof comprising the STING promoter is obtained from a sample from a subject. In some instances, the sample comprises a malignant cell or tissue. In some instances, the sample comprises a tumor and/or immune cell. Different types of tissue correspond to different types of cells (e.g., brain, Schwann cells, liver, and the like), but also healthy and/or tumor cells, including tumor cells at various stages of neoplasia and metastasized or displaced tumor cells. In some embodiments, a tissue sample further encompasses a clinical sample, and also includes cells in culture, cell supernatants, tissue, organ, and the like.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a human. In other embodiments, the subject comprises a human subject suffering from, or believed to be suffering from, a STING-silenced cancer.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like. As is known in the art, a cancer is generally considered as uncontrolled cell growth. The methods of the present disclosure can be used to treat any cancer, and any metastases thereof. In some embodiments, the cancer being treated is glioblastoma, also known as glioblastoma multiforme (GBM). In some embodiments, the disease comprises a tumor of neural crest and/or neuroectodermal origin, whether cranial or extra-cranial.

Glioblastoma is an often-fatal brain malignancy that is the most common primary brain tumor. The incidence of GBM is 2.5 per 100,000 adults, or 8,250 diagnoses in the US a year. Glioblastoma is a brain cancer having a poor prognosis. The median survival is just over 14 months and the recurrence rate for this disease is more than 90%. (Lynes, J., Sanchez, V., Dominab, G, Nwankwo, A., & Nduom, E. (2018). Current options and future directions in immune therapy for glioblastoma. Frontiers in oncology, 8, 578. This is a disease with a clear need for new treatment strategies. Standard of care for newly diagnosed GBM includes maximal surgical resection, followed by treatment with a DNA alkylating agent, temozolomide, and radiation. (Xiao, Q., Yang, S., Ding, G., & Luo, M. (2018). Anti-vascular endothelial growth factor in glioblastoma: a systematic review and meta-analysis. Neurological Sciences, 39(12), 2021-2031) Currently, there is no standard of care for recurrent GBM disease. The anti-VEGF therapies such as, bevacizumab have been shown to improve progression free survival but not overall survival. (Xiao, Q., Yang, S., Ding, G., & Luo, M. (2018). Anti-vascular endothelial growth factor in glioblastoma: a systematic review and meta-analysis. Neurological Sciences, 39(12), 2021-2031) Immune therapies such as CAR-T therapy against CD133 and checkpoint inhibitors are in clinical trials for the treatment of GBM. (Lynes, J., Sanchez, V., Dominah, G., Nwankwo, A., & Nduom, E. (2018). Current options and future directions in immune therapy for glioblastoma. Frontiers in oncology, 8, 578.) Glioblastoma can be classified according to isocitrate dehydrogenase (IDH) mutation. If wild-type, then called primary and if IDH-mutant is called secondary

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, “treatment,” “therapy” and/or “therapy regimen” refer to the clinical intervention made in response to a disease, disorder or physiological condition manifested by a patient/subject or to which a patient/subject may be susceptible. The aim of treatment includes the alleviation or prevention of symptoms, slowing or stopping the progression or worsening of a disease, disorder, or condition and/or the remission of the disease, disorder or condition. As used herein, the term “appropriate anti-cancer therapy” includes those treatments that are effective in the treatment of cancers of neural crest origin. Examples of these of such therapy include, but are not limited to, surgery (e.g., curative resection, transplantation, etc.), radiation, chemotherapy (e.g., cisplatin, doxorubicin, fluoropyrimidine, gemcitabine, irinotecan, mitoxantrone, oxaliplatin, thalidomide, or a combination thereof. In some cases, the agent for the targeted therapy comprises axitinib, bevacizumab, cetuximab, erlotinib, ramucirumab, regorafenib, sorafenib, sunitinib, a thymidine kinase (TK) inhibitor, or a combination thereof), targeted drug therapy, and the like. In some instances, the treatment comprises transcatheter arterial chemoembolization, radiofrequency ablation, or brachytherapy. In some embodiments, the treatment comprises surgery. In some cases, surgery comprises curative resection.

As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

In some embodiments, the therapeutic agent or immunotherapy comprise an immune checkpoint inhibitor. Immune checkpoint inhibitors which may be used according to the invention are any that disrupt the inhibitory interaction of cytotoxic T cells and tumor cells. Inhibitors may target any immune checkpoint known in the art, including but not limited to, CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, and the B-7 family of ligands. Non-limiting examples of immune checkpoint inhibitors include but are not limited to anti-PD-1 antibody, anti-PD-L1 antibody, anti-CTLA4 antibody, antiLAG-3 antibody, and/or anti-TIM-3 antibody. Approved checkpoint inhibitors in the U.S. include ipimilumab, pembrolizumab, and nivolumab. The inhibitor need not be an antibody but can be a small molecule or other polymer. If the inhibitor is an antibody then it can be a polyclonal, monoclonal, fragment, single chain, or any other antibody variant construct which retains antigen binding.

In some embodiments, a number of methods are utilized to measure, detect, determine, identify, and characterize the methylation status/level of a gene or a biomarker (e.g., CpG island-containing region/fragment such as that associated with cg16983159) in identifying a subject as having a STING-silenced tumor, the prognosis of a subject having such a cancer, the progression or regression of a said cancer in subject in the presence of a therapeutic agent, and/or providing an indication for susceptibility to an anti-cancer therapy for a STING-silenced tumor in a subject.

In some embodiments, a biomarker (or an epigenetic marker), such as cg16983159, is obtained from a tissue sample. In some instances, a tissue corresponds to any cell(s). Different types of tissue correspond to different types of cells (e.g., liver, lung, blood, connective tissue, and the like), but also healthy cells vs. tumor cells or to tumor cells at various stages of neoplasia, or to displaced malignant tumor cells. In some embodiments, a tissue sample further encompasses a clinical sample, and also includes cells in culture, cell supernatants, organs, and the like. Samples also comprise fresh-frozen and/or formalin-fixed, paraffin-embedded tissue blocks, such as blocks prepared from clinical or pathological biopsies, prepared for pathological analysis or study by immunohistochemistry.

In some embodiments, a biomarker (or an epigenetic marker), such as cg16983159, is methylated or unmethylated in a normal sample (e.g., normal or control tissue without disease, or normal or control body fluid, stool, blood, serum, amniotic fluid), most importantly in healthy stool, blood, serum, amniotic fluid or other body fluid. In other embodiments, a biomarker (or an epigenetic marker) is hypomethylated or hypermethylated in a sample from a patient having or at risk of a disease (e.g., one or more indications described herein); for example, at a decreased or increased (respectively) methylation frequency of at least about 50%, at least about 60%, at least about 70%, at least about 75%, at least about 80%, at least about 85%, at least about 90%, at least about 95%, or about 100% in comparison to a normal sample. In some embodiments, a sample is also hypomethylated or hypermethylated in comparison to a previously obtained sample analysis of the same patient having or at risk of a disease (e.g., one or more indications described herein), particularly to compare progression of a disease.

In some embodiments, a set of epigenetic markers or methylation sites, such as a biomarker described above. In some instances, a set of methylation sites that corresponds to the entire promoter of a STING gene of a tumor cell or a population of cells in a tumor or malignant tissue. In some cases, a set of methylation sites is determined using a tumor tissue (e.g. with or without immune cells) or cell-free (or protein-free) tumor DNA in a biological sample.

In some embodiments, DNA (e.g., genomic DNA such as extracted genomic DNA or treated genomic DNA) is isolated by any means standard in the art, including the use of commercially available kits. Briefly, wherein the DNA of interest is encapsulated in by a cellular membrane the biological sample is disrupted and lysed by enzymatic, chemical or mechanical means. In some cases, the DNA solution is then cleared of proteins and other contaminants e.g. by digestion with proteinase K. The DNA is then recovered from the solution. In such cases, this is carried out by means of a variety of methods including salting out, organic extraction or binding of the DNA to a solid phase support. In some instances, the choice of method is affected by several factors including time, expense and required quantity of DNA.

Wherein the sample DNA is not enclosed in a membrane (e.g. circulating DNA from a cell free sample such as blood or urine) methods standard in the art for the isolation and/or purification of DNA are optionally employed). Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. In some cases, the person skilled in the art also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

### B. Detecting Methylation of a CpG in a TMEM173 (STING) Promoter

Methylation status of a gene or DNA molecule in a sample can be detected using a method known in the art and/or described herein. A variety of methylation detection techniques known in the art may be used to practice the methods disclosed herein. Any assay known to the skilled worker that provides for determination of the methylation state of one or a plurality of CpG sites within a STING promoter of a tissue sample, or derivative thereof, may be suitable for use in any method of the disclosure. In addition, these methods may be used for absolute or relative quantification of a methylated nucleic STING promoter or a subregion thereof or a specific CpG site(s) therein.

In some embodiments, DNA methylation is detected and/or quantified using techniques known to the skilled worker. Non-limiting examples of such methods include bisulfite sequencing, bisulfite conversion and DNA sequencing (e.g. via PCR or cloning and via methylation-specific PCR or bisulfite pyrosequencing), affinity capture specific to a methylated variant, and differential restriction fragment analyses. In certain embodiments, the method utilizes microfluidic techniques, e.g. using a ChIP then quantitative PCR (qPCR) approach. Methylation status can be determined at the level of a single cell or a collection of cells. In certain instances, the collection of cells may include both tumor cells and non-tumor cells, such as non-neoplastic immune cells.

For affinity capture, typically methylated DNA is immunoprecipitated using a method commonly known as methylated DNA immunoprecipitation (Me-DIP) that uses a methyl-DNA specific antibody or methyl capture using methyl-CpG binding domain (MBD) protein. Devices and setups for providing semi-automated bisulfite conversion are known in the art (see e.g. Stark A, et al. Biomed Microdevices 18, 5 (2016); Zhang H et al., Biosens Bioelectron 42, 503-11 (2013)).

Methods of bisulfite-based detection of methylated DNA can provide high sensitivity of detection, such as methylomic profiles at single-nucleotide resolution. Microfluidic technology is often used to perform low-input, rapid, and automated bisulfite treatment. Site-specific analysis, such as methylation-specific PCR (MS-PCR) may be used as a fast diagnostic tool for detecting DNA methylation. On-chip bisulfite conversion methods are known in the art (see e.g. Yoon J et al., Lab on a Chip 15, 35303539 (2015)) Devices for droplet in oil bisulfite conversion are known in the art (see e.g. Zhang Y et al., Lab on a Chip, 9, 1059-64 (2009)). Devices for microfluidic PCR-bisulfite conversion are known in the art (see e.g. Heng J et al., Oncotarget 8, 25442-54 (2017)).

Devices and setups for providing differential melting temperature detection of methylated DNA are known in the art (see e.g. U.S. Pat. No. 8,911,937). For example, DNA methylation may be detected via capillary electrophoresis devices (see e.g. Fang G et al., Nature Biotechnology 30, 1232 (2012); Zhang H et al., Lab Chip 7, 1162-70 (2007)). Devices and setups for providing differential melting temperature detection of methylated DNA are known in the art (see e.g. Liu F et al., Lab on a Chip 18, 514-21 (2018); O'Keefe C et al., Sci Adv 4, eaat6459 (2018); Pisanic T et al., Nucleic Acids Res 43, e154 (2015)). Devices are available for performing High Resolution Melting (HRM) detecting and quantification of methylated DNA (see e.g. Athamanolap P et al., Jala-J Lab Autom 19, 304-12 (2014)). HRM may also be combined with other amplification techniques such as pyrosequencing as described by Candiloro et al., 2011, Epigenetics 6(4) 500-507). Devices and setups for performing HYPER-Melt or DREAMing (digital restriction enzyme analysis of methylation) are known in the art.

Disclosed herein is a method of detecting the methylation status of one or more CpGs in the STING promoter of a subject, comprising processing a biological sample obtained from the subject to extract DNA nucleic acids. After the nucleic acids have been extracted, a DNA methylation analysis of the STING promoter is carried out by any means known in the art.

Methods of detecting the methylation status of one or more CpGs in the STING promoter of a subject are provided. The methods include (a) processing a biological sample obtained from the subject with a deaminating agent to generate treated DNA comprising deaminated nucleotides; and (b) detecting the methylation status of a STING promoter. In some instances, the method further comprises contacting the treated DNA with a probe or probes that hybridize under high stringency conditions to the STING promoter to generate an amplified product, and analyzing the amplified product to generate a methylation profile of the STING promoter or a subregion thereof.

The methods may comprise treating a target nucleic acid molecule with a reagent that modifies nucleotides of the target nucleic acid molecule as a function of the methylation state of the target nucleic acid molecule, amplifying treated target nucleic acid molecule, fragmenting amplified target nucleic acid molecule, and detecting one or more amplified target nucleic acid molecule fragments, and based upon the fragments, such as size and/or number thereof, identifying the methylation state of a target nucleic acid molecule, or a nucleotide locus in the nucleic acid molecule, or identifying the nucleic acid molecule or a nucleotide locus therein as methylated or unmethylated.

The methods may comprise treating a target nucleic acid molecule with a reagent that modifies nucleotides of the target nucleic acid molecule as a function of the methylation state of the target nucleic acid molecule, amplifying treated target nucleic acid molecule, fragmenting amplified target nucleic acid molecule, and detecting one or more amplified target nucleic acid molecule fragments, and based upon the fragments, such as size and/or number thereof, identifying the methylation state of a target nucleic acid molecule, or a nucleotide locus in the nucleic acid molecule, or identifying the nucleic acid molecule or a nucleotide locus therein as methylated or unmethylated. Fragmentation can be performed, for example, by treating amplified products under base specific cleavage conditions. Detection of the fragments can be affected by measuring or detecting a mass of one or more amplified target nucleic acid molecule fragments, for example, by mass spectrometry such as MALDI-TOF mass spectrometry. Detection also can be affected, for example, by comparing the measured mass of one or more target nucleic acid molecule fragments to the measured mass of one or more reference nucleic acid, such as measured mass for fragments of untreated nucleic acid molecules. In an exemplary method, the reagent modifies unmethylated nucleotides, and following modification, the resulting modified target is specifically amplified. In some embodiments, the methods for determining the methylation state of a STING promoter may include treating a target nucleic acid molecule with a reagent that modifies a selected nucleotide as a function of the methylation state of the selected nucleotide to produce a different nucleotide. Embodiments, the reagent that modifies unmethylated cytosine to produce uracil is bisulfite. In certain embodiments, the methylated or unmethylated nucleic acid base is cytosine. In another embodiment, a non-bisulfite reagent modifies unmethylated cytosine to produce uracil.

One skilled in the art will recognize that amplification can be accomplished by any known method, such as by polymerase chain reaction (PCR), isothermal amplification, ligase chain reaction (LCR), Q-replicas amplification, rolling circle amplification, transcription amplification, self-sustained sequence replication, nucleic acid sequence-based amplification (NASBA). In some embodiments, a branched-DNA assay is used to qualitatively demonstrate the presence of a sequence that represents a particular methylation pattern. In some embodiments, the nucleic acids are amplified and methylation detected using PCR. Various PCR techniques known in the art may be used in the method, for example, reverse transcription PCR, ligation mediated PCR, digital PCR (dPCR), and droplet digital PCR (ddPCR).

In some instances, quantitative amplification methods (e.g., qPCR or quantitative linear amplification) are used to quantify the amount of intact DNA within a locus of interest (e.g. a STING promoter region) flanked by amplification primers following restriction digestion. Non-limiting example of methods of quantitative amplification are described in U.S. Pat. Nos. 5,972,602; 6,033,854; 6,180,349; Gibson et al, 6 Genome Research 995-1001 (1996); Deiman B, et al., 20(2) Mol. Biotechnol. 163-79 (2002); and DeGraves, et al, 34(1) Biotechniques 106-15 (2003).

The methods provided herein further provide for the use of a sequence specific probe. In some embodiments, the probe comprises a DNA probe, RNA probe, or a combination thereof. In some instances, a probe comprises natural nucleic acid molecules and non-natural nucleic acid molecules. In some cases, a probe comprises a labeled probe, such as for example, fluorescently labeled probe or radioactively labeled probe. In some instances, a probe correlates to a CpG site. In some instances, a probe is utilized in a next generation sequencing reaction to generate a CpG methylation data. In further instances, a probe is used in a solution-based next generation sequencing reaction to generate a CpG methylation data.

The skilled worker may use any pyrosequencing technique known in the art in the methods of the disclosure. Devices for pyrosequencing and compatible methylation specific reagents are commercially available. In some embodiments, methylation is detected via pyrosequencing method wherein genomic DNA from the sample or a amplified product(s) thereof are immobilized to a solid support, hybridized with a sequencing primer, incubated with DNA polymerase, ATP sulfurylase, luciferase, apyrase, adenosine 5′ phosphosulfate and luciferin; and wherein different nucleotide solutions are sequentially added and removed in a controlled fashion while a chemiluminescent signal is recorded. An example of a method of pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a multiwell solid support; and sequencing amplified nucleotide sequences using a pyrosequencing technique (see e.g. Nakano et al, J. Biotech. 102, 117-124 (2003)).

In some embodiments, a next generation sequencing (NGS) technology known in the art is used to detect or quantify DNA methylation. Suitable next generation sequencing technologies are also widely available and considered within the scope of the present disclosure. Non-limiting examples of NGS include the 454 Life Sciences platform (Roche, Branford, CT); lllumina's Genome Analyzer, GoldenGate Methylation Assay, or Infinium Methylation Assays, e.g., Infinium HumanMethylation 27K BeadArray or VeraCode GoldenGate methylation array (Illumina, San Diego, CA); QX200™ Droplet Digital™ PCR System from Bio-Rad; or DNA Sequencing by Ligation, SOLiD System (Applied Biosystems/Life Technologies, Waltham, MA); the Helicos True Single Molecule DNA sequencing technology (see e.g. Harris et al, 2008 Science, 320, 106-109), the single molecule, real-time (SMRT™) technology of Pacific Biosciences (Menlo Park, CA), and solid state nanopore sequencing (Soni and Meller, 2007, Clin. Chem. 53, 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). Typically, NGS technologies allow for sequencing of clonally expanded or non-amplified single molecules of DNA fragments. In some instances, such NGS platforms provide specialized methods of (i) sequencing by ligation of dye-modified probes (including cyclic ligation and cleavage), (ii) pyrosequencing, and (iii) single-molecule sequencing.

In some embodiments, differential methylation is detected using an invasive cleavage reaction (see e.g. U.S. Pat. Nos. 8,916,344; 7,011,944).

In some instances, once the nucleic acids have been extracted, methylation analysis is carried out by any means known in the art. A variety of methylation analysis procedures are known in the art and may be used to practice the methods disclosed herein. These assays allow for determination of the methylation state of one or a plurality of CpG sites within a tissue sample. In addition, these methods may be used for absolute or relative quantification of methylated nucleic acids. Such methylation assays involve, among other techniques, two major steps. The first step is a methylation specific reaction or separation, such as (i) bisulfite treatment, (ii) methylation specific binding, or (iii) methylation specific restriction enzymes. The second major step involves (i) amplification and detection, or (ii) direct detection, by a variety of methods such as (a) PCR (sequence-specific amplification), (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

Restriction enzyme digestion of PCR products amplified from bisulfite-converted DNA may be used, e.g., the method described by Sadri and Hornsby (1996, Nucl. Acids Res. 24:5058-5059), or COBRA (Combined Bisulfite Restriction Analysis) (Xiong and Laird, 1997, Nucleic Acids Res. 25:2532-2534).

In some embodiments, the methylation profile of selected CpG sites is determined using methylation-specific PCR (MSP) (see e.g. Herman et al, 1996, Proc. Nat. Acad. Sci. USA, 93, 9821-9826; U.S. Pat. Nos. 5,786,146; 6,017,704; 6,200,756; 6,265,171; 8,969,046).

In some embodiments, the methylation profile of selected CpG sites is determined using a MethyLight and/or Heavy Methyl Method, which are high-throughput quantitative methylation assays based on real-time PCR (see e.g. Eads, C. A. et al, 2000, Nucleic Acid Res. 28, e 32; Cottrell et al, 2007, J. Urology 177, 1753, U.S. Pat. No. 6,331,393). Quantitative MethyLight uses bisulfite to convert genomic DNA and the methylated sites are amplified using PCR with methylation independent primers. Detection probes specific for the methylated and unmethylated sites with two different fluorophores provides simultaneous quantitative measurement of the methylation. The Heavy Methyl technique begins with bisulfite conversion of DNA. Next specific blockers prevent the amplification of unmethylated DNA. Methylated genomic DNA does not bind the blockers and their sequences will be amplified. The amplified sequences are detected with a methylation specific probe.

In some embodiments, the methylation is detecting using a Ms-SNuPE method, which is a quantitative method for assessing methylation differences at specific CpG sites based on bisulfite conversion followed by single-nucleotide primer extension.

In some embodiments, a 5-methyl cytidine antibody to bind and precipitate methylated DNA, such as using a commercially available antibody (see e.g. Pelizzola et al, 2008, Genome Res. 18, 1652-1659; O'Geen et al, 2006, BioTechniques 41(5), 577-580, Weber et al, 2005, Nat. Genet. 37, 853-862; Horak and Snyder, 2002, Methods Enzymol, 350, 469-83; Lieb, 2003, Methods Mol Biol, 224, 99-109). The Roche® NimbleGen® microarrays including the Chromatin Immunoprecipitation-on-chip (ChIP-chip) or methylated DNA immunoprecipitation-on-chip (MeDIP-chip). Another technique is methyl-CpG binding domain column/segregation of partly melted molecules (MBD/SPM) (see e.g. Shiraishi et al, Proc. Natl. Acad. Sci. USA 96(6):2913-2918 (1999)) In some embodiments, methylation status is detected using a Methyl-CpG immunoprecipitation (MCIP) method wherein the methyl binding domain of M1BD2 is fused to the Fc fragment of an antibody (MBD-FC). The strength of this protein-DNA interaction is defined by the level of DNA methylation. After binding genomic DNA, eluate solutions of increasing salt concentrations can be used to fractionate non-methylated and methylated DNA allowing for a more controlled separation.

In some embodiments, the method comprises detecting a differential restriction map based on a methyl-sensitive restriction enzyme. For example, intact DNA may be amplified after restriction enzyme digestion, thereby only amplifying certain STING promoter DNA region(s) that was not cleaved by the restriction enzyme in the area amplified (see, e.g., U.S. Pat. Nos. 7,459,274; 7,901,880; 7,910,296). One of skill in the art will appreciate that any methylation-dependent restriction enzyme may be suitable for use with one or more methods described herein. Further, one of skill in the art will appreciate that some methylation-sensitive restriction enzymes are blocked by methylation of bases on one or both strands of a double stranded DNA encompassing of their recognition sequence, while other methylation-sensitive restriction enzymes are blocked only by methylation on both strands, but can cleave a DNA molecule when the recognition site is hemi-methylated.

In some embodiments, methylation is detected using a method described in, e.g., U.S. Pat. Nos. 7,553,627 and 6,331,393.

### C. CpG Methylation Data Analysis

Once it is determined that a STING promoter in a sample is methylated or unmethylated, additional data analysis can be used to further elucidate details of STING promoters in a sample. For example, data analysis can be used to determine methylation status of specific CpG sites, specific methylation patterns over a promoter region, and/or tumor compositions wherein different cells comprise different STING promoter methylation patterns. In some instances, a control biological sample or control STING promoter DNA molecule is used in a quantitative method as a standard for normalization or calculating relative methylation values.

In some embodiments, the methylation values measured for biomarkers of a biomarker panel (e.g. two or more individual CpG sites in the STING promoter) are mathematically combined and the combined value is correlated to the underlying diagnostic question, such as to produce a differential methylation correlation score at a given statistical significance level. In some instances, methylated biomarker values are combined by any appropriate mathematical method known in the art, such as using a discriminant analysis (DA) (e.g., linear-, quadratic-, regularized-DA), discriminant functional analysis (DFA), fuzzy logic based methods, generalized additive models, generalized linear models (e.g., logistic regression), kernel methods (e.g., SVM), multidimensional scaling (MDS), neural networks and genetic algorithms based methods, nonparametric methods (e.g., k-nearest-neighbor classifiers), partial least squares (PLS), principal components based methods (e.g., SIMCA), and tree-based methods (e.g., logic regression, cart, random forest methods, boosting/bagging methods). The skilled worker will be able to selecting an appropriate method to evaluate an epigenetic marker or biomarker combination described herein.

In some embodiments, a diagnostic test to correctly predict status is measured using the sensitivity of the assay, the specificity of the assay, or the area under a receiver operated characteristic (“ROC”) curve. The greater the area under the ROC curve, for example, the more accurate or powerful the predictive value of the test. In some instances, sensitivity of the assay is the percentage of true positives that are predicted by a test to be positive, while specificity of the assay is the percentage of true negatives that are predicted by a test to be negative. Other useful measures of the utility of a test include positive predictive value and negative predictive value. Positive predictive value is the percentage of subjects who test positive that are actually positive. Negative predictive value is the percentage of subjects who test negative that are actually negative. In some embodiments, one or more of the biomarkers disclosed herein show a statistical difference in different samples of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

In some embodiments, the correlation of a combination of biomarkers in a sample is compared, for example, to a predefined set of biomarkers. In some embodiments, the measurement(s) is then compared with a relevant diagnostic amount(s), cut-off(s), or multivariate model scores that distinguish between therapeutic outcomes. The skilled worker will be able to adjust the diagnostic cut-off(s) used in an assay to increase sensitivity or specificity of the diagnostic assay. In some embodiments, the particular diagnostic cut-off is determined by measuring the amount of methylation of a specific CpG island or site in a statistically significant number of samples from patients to determine diagnostic cut-offs which match the desired levels of specificity and sensitivity.

“Contacting” as used herein, e.g., as in “contacting a sample” refers to contacting a sample directly or indirectly in vitro, ex vivo, or in vivo (i.e. within a subject as defined herein). Contacting a sample may include addition of a compound to a sample, or administration to a subject. Contacting encompasses administration to a solution, cell, tissue, mammal, subject, patient, or human. Further, contacting a cell includes adding an agent to a cell culture.

As used herein, the term “correlates” as between a specific diagnosis or a therapeutic outcome of a sample or of an individual and the changes in methylation state of a nucleic acid target gene region refers to an identifiable connection between a particular diagnosis or therapy of a sample or of an individual and its methylation state.

As used herein, the term “biomarker” or “epigenetic marker” refers to a naturally occurring biological molecule present in a subject at varying concentrations useful in predicting the risk or incidence of a disease or a condition, such as a STING-silenced cancer. For example, the biomarker can be a protein present in higher or lower amounts in a subject at risk for metastatic pancreatic cancer. The biomarker can include nucleic acids, ribonucleic acids, or a polypeptide used as an indicator or marker for metastatic pancreatic cancer in the subject. In other embodiments, the biomarkers may comprise a methylation status/pattern. DNA methylation silences expression of tumor suppression genes and presents itself as one of the first neoplastic changes. Methylation patterns found in neoplastic tissue and plasma demonstrate homogeneity, and in some instances are utilized as a sensitive diagnostic marker. As used herein, a “methylation” or “methylation state” or “methylation pattern” or “methylation status” that correlates with a disease, disease outcome or outcome of a treatment regimen refers to a specific methylation state of a nucleic acid target gene region or nucleotide locus (e.g., cg16983159) that is present or absent more frequently in subjects with a known disease, disease outcome or outcome of a treatment regimen, relative to the methylation state of a nucleic acid target, gene region or nucleotide locus than otherwise occur in a larger population of individuals (e.g., a population of all individuals).

### D. Kits

Another aspect of the present disclosure provides a kit for the prognosis, diagnosis, or prediction of a STING-silenced tumor comprising, consisting of, or consisting essentially of: (a) a means for analyzing the methylation pattern of marker cg16983159; (b) a control; and (c) instructions for use.

In some instances, the kit comprises a plurality of primers or probes to detect or measure the methylation status/levels of one or more samples. Such kits comprise, in some instances, at least one polynucleotide that hybridizes to at least one of the methylation marker sequences described herein (e.g., cg16983159) and at least one reagent for detection of gene methylation. Reagents for detection of methylation include, e.g., sodium bisulfite, polynucleotides designed to hybridize to sequence that is the product of a marker sequence if the marker sequence is not methylated (e.g., containing at least one C-U conversion), and/or a methylation-sensitive or methylation-dependent restriction enzyme. In some cases, the kits provide solid supports in the form of an assay apparatus that is adapted to use in the assay. In some instances, the kits further comprise detectable labels, optionally linked to a polynucleotide, e.g., a probe, in the kit.

In some embodiments, the kits comprise one or more (e.g., 1, 2, 3, 4, or more) different polynucleotides (e.g., primers and/or probes) capable of specifically amplifying at least a portion of a DNA region of a biomarker described herein. Optionally, one or more detectably labeled polypeptides capable of hybridizing to the amplified portion are also included in the kit. In some embodiments, the kits comprise sufficient primers to amplify 2, 3, 4, 5, 6, 7, 8, 9, 10, or more different DNA regions or portions thereof, and optionally include detectably-labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters (e.g., oligonucleotides that can be ligated or otherwise linked to genomic fragments) for whole genome amplification, and polynucleotides (e.g., detectably-labeled polynucleotides) to quantify the presence of the converted methylated and or the converted unmethylated sequence of at least one cytosine from a DNA region of an epigenetic marker described herein.

In some embodiments, the kits comprise methylation sensing restriction enzymes (e.g., a methylation-dependent restriction enzyme and/or a methylation-sensitive restriction enzyme), primers and adapters for whole genome amplification, and polynucleotides to quantify the number of copies of at least a portion of a DNA region of an epigenetic marker described herein.

In some embodiments, the kits comprise a methylation binding moiety and one or more polynucleotides to quantify the number of copies of at least a portion of a DNA region of a marker described herein. A methylation binding moiety refers to a molecule (e.g., a polypeptide) that specifically binds to methyl-cytosine.

Examples include restriction enzymes or fragments thereof that lack DNA cutting activity but retain the ability to bind methylated DNA, antibodies that specifically bind to methylated DNA, etc.).

In some embodiments, the kit includes a packaging material. As used herein, the term “packaging material” can refer to a physical structure housing the components of the kit. In some instances, the packaging material maintains sterility of the kit components, and is made of material commonly used for such purposes (e.g., paper, corrugated fiber, glass, plastic, foil, ampules, etc.). Other materials useful in the performance of the assays are included in the kits, including test tubes, transfer pipettes, and the like. In some cases, the kits also include written instructions for the use of one or more of these reagents in any of the assays described herein.

In some embodiments, kits also include a buffering agent, a preservative, or a protein/nucleic acid stabilizing agent. In some cases, kits also include other components of a reaction mixture as described herein. For example, kits include one or more aliquots of thermostable DNA polymerase as described herein, and/or one or more aliquots of dNTPs. In some cases, kits also include control samples of known amounts of template DNA molecules harboring the individual alleles of a locus. In some embodiments, the kit includes a negative control sample, e.g., a sample that does not contain DNA molecules harboring the individual alleles of a locus. In some embodiments, the kit includes a positive control sample, e.g., a sample containing known amounts of one or more of the individual alleles of a locus.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.” Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The skilled worker will be able to determine the sequence identity of a given DNA or protein sequence by comparison with polynucleotides or polypeptide sequences of the same size, or by computer homology programs known in the art for alignment. A non-limiting example of a program known in the art is the GAP program (Wisconsin Sequence Analysis Package, Version 8 for UNIX, Genetics Computer Group,) that uses the algorithms of Smith T, Waterman M, Adv. Appl. Math. 2: 482-9 (1981) and uses default settings.

Unless otherwise defined, all technical terms used herein have the same meaning as commonly understood by one of ordinary skill in the art to which this disclosure belongs.

Another aspect of the present disclosure provides all that is described and illustrated herein.

The following Examples are provided by way of illustration and not by way of limitation.

### EXAMPLES

### Example 1—STING Promoter Methylation at a Single Site cg16983159 Leads to Epigenetic Silencing in Glioblastoma and Other Tumors of Neural Crest Origin

In this study, STING DNA methylation, expression, downstream dysregulation was investigated in glioblastoma patient samples and cell culture systems. One specific CpG site was found in the STING promoter that is universally highly methylated in glioblastoma cells and is associated with low or absent STING RNA expression. STING methylation was also analyzed in normal brain samples and across a spectrum of non-cancerous cerebral diseases, and it was discovered that STING promoter hypermethylation is not unique to GBM, but rather is a common finding across these diverse brain states. Finally, STING methylation was examined across cancer and STING promoter hypermethylation at this site is generally rare in extracranial cancers, but STING promoter hypermethylation is also found in other primary brain tumors and extracranial tumors predominantly of neuroectodermal embryologic origin. In contrast, amongst systemic tumors that generally show low overall STING methylation, a subset of samples typically exhibits STING promoter hypermethylation. We hypothesized that such STING-silenced tumors will behave similarly to brain tumors and may not respond as robustly to traditional anti-cancer therapies, including immunotherapy. Collectively, these results provide mechanistic insights into the immune microenvironment in which glioblastoma and related tumors develop.

### A. Results

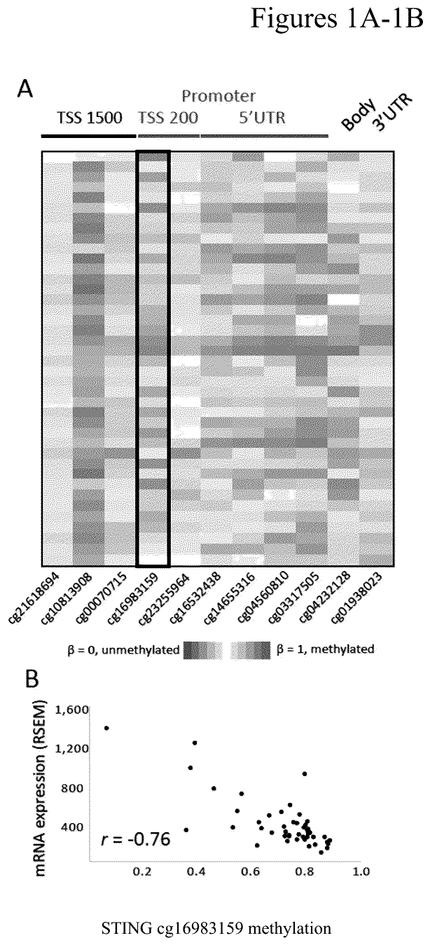

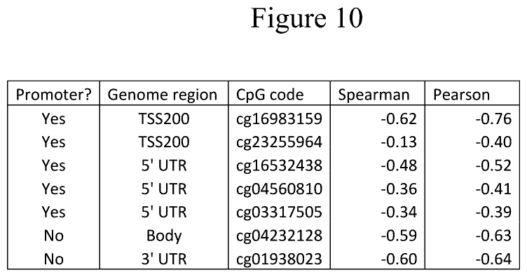

STING expression is silenced by promoter hypermethylation in glioblastoma and across a spectrum of neurologic disease. Illumina 450 k and EPIC methylation arrays were performed on glioblastoma patient samples (450 k cohort described previously) and analyzed the methylation values across all 11 probes within the STING gene (FIG. 1A). The STING promoter is generally unmethylated (FIG. 1A) with the notable exception of site cg16983159. TCGA data was analyzed to determine if methylation was correlated with RNA expression. In the GBM pan-cancer TCGA cohort, 46 samples included both methylation array and RNA expression data. Methylation array data are available for 7 of the 11 Illumina 450K array probes within the STING gene. Methylation values for each of these 7 probes demonstrate variable negative correlations with mRNA expression, with the strongest negative correlation being with probe cg16983159 (FIG. 1B, FIG. 10). This suggests that DNA methylation in the region of cg16983159 may suppress STING mRNA expression.

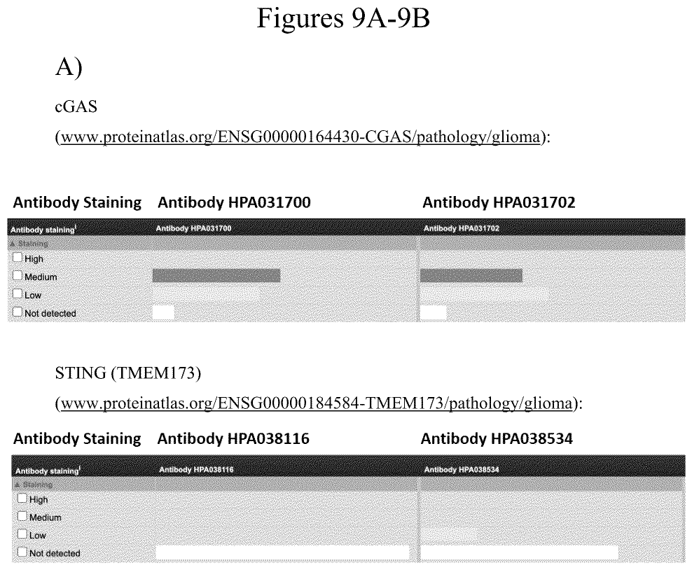

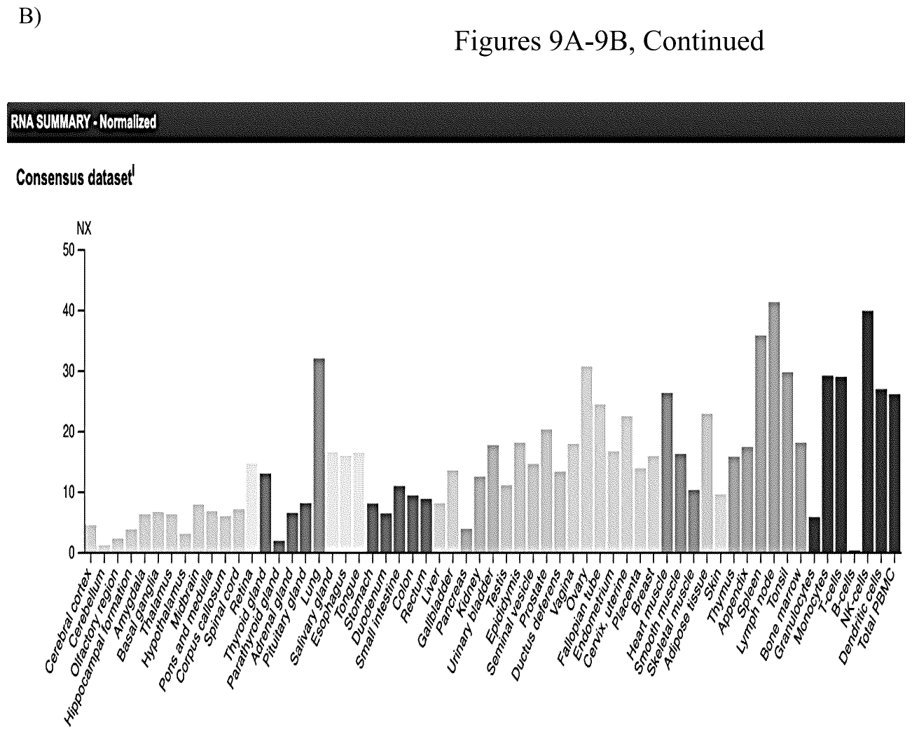

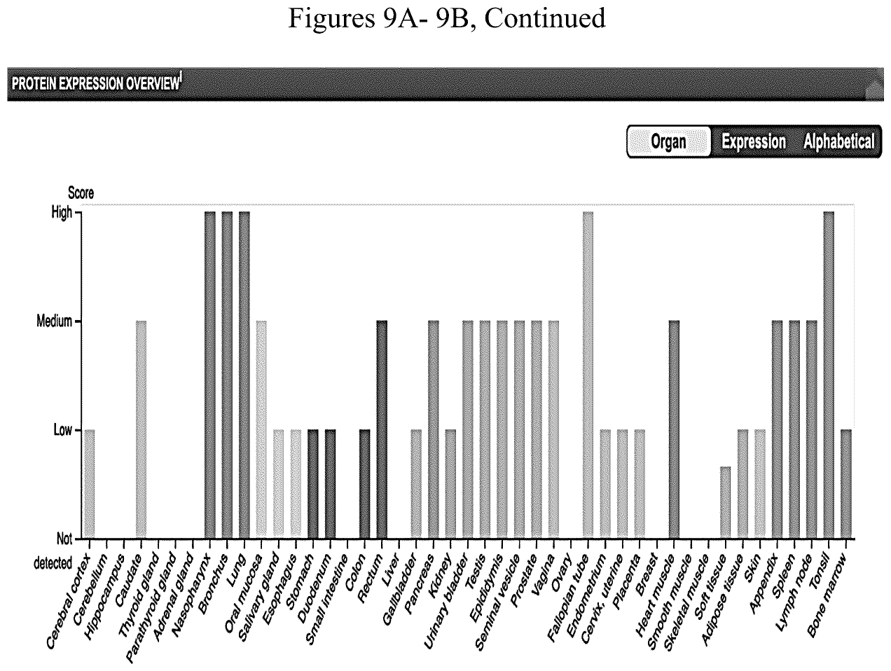

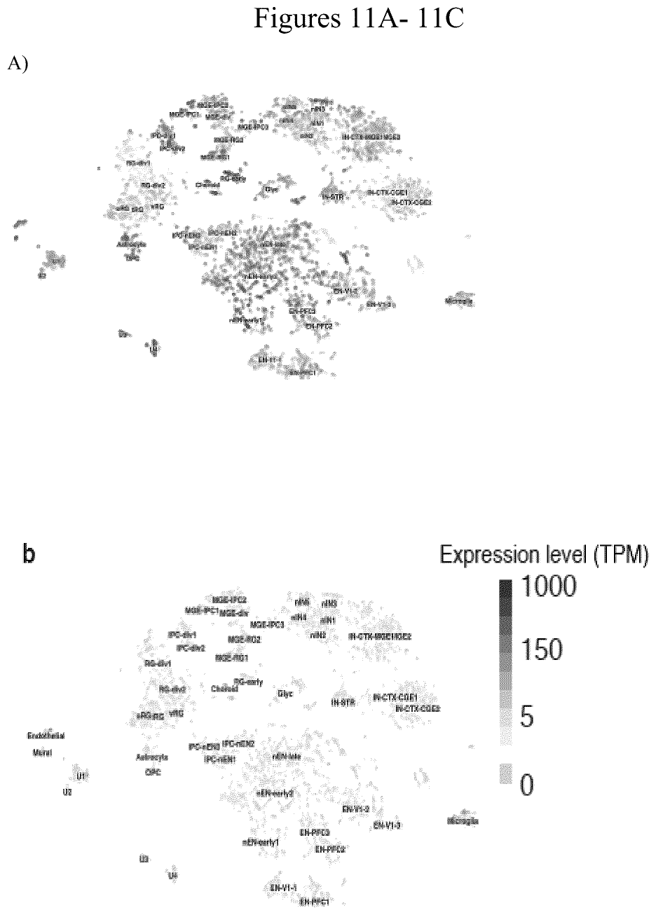

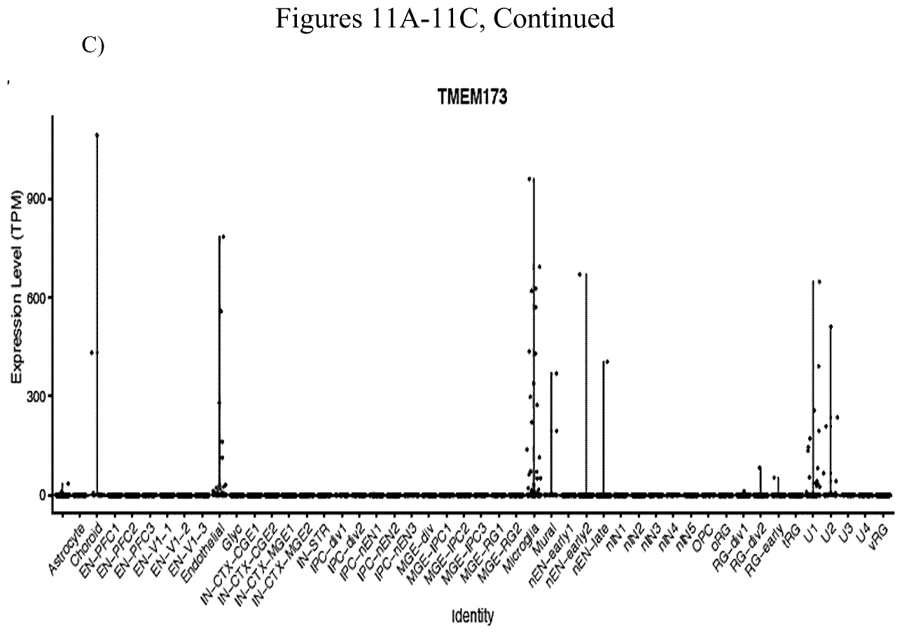

The STING promoter cg16983159 site is hypermethylated in the non-cancerous brain. It was next asked whether the pattern of STING promoter cg16983159 hypermethylation and gene expression silencing is specific to glioblastoma or if it is also present in normal brain parenchyma and other non-cancerous neurologic disease states. Bulk methylation array data was analyzed from patients without known neurologic disease, as well as those with Alzheimer, Parkinson, Multiple Sclerosis, Schizophrenia, and Bipolar diseases. In all cases, the cg16983159 STING promoter site is highly methylated. Correspondingly, data summarized in the Human Protein Atlas show that STING RNA expression in the normal brain is amongst the lowest of all tissue types. Similarly, STING protein expression is also generally low in the normal human brain (FIG. 9B). This silencing of STING expression seems to occur early in development, as scRNA expression data from human and mouse fetuses show an absence of STING expression in neurons and glia (FIG. 11). STING is only expressed in immune and endothelial cells.

Pan-cancer analysis reveals distinct patterns of STING promoter methylation and is most commonly represented in tumors derived from neuroectoderm. It was queried whether STING epigenetic silencing is seen in other brain cancers as well as in systemic cancers and the normal tissues in which they arise. Published Illumina 450K methylation array datasets were analyzed from TCGA and publicly available datasets deposited in the Gene Expression Omnibus (GEO).

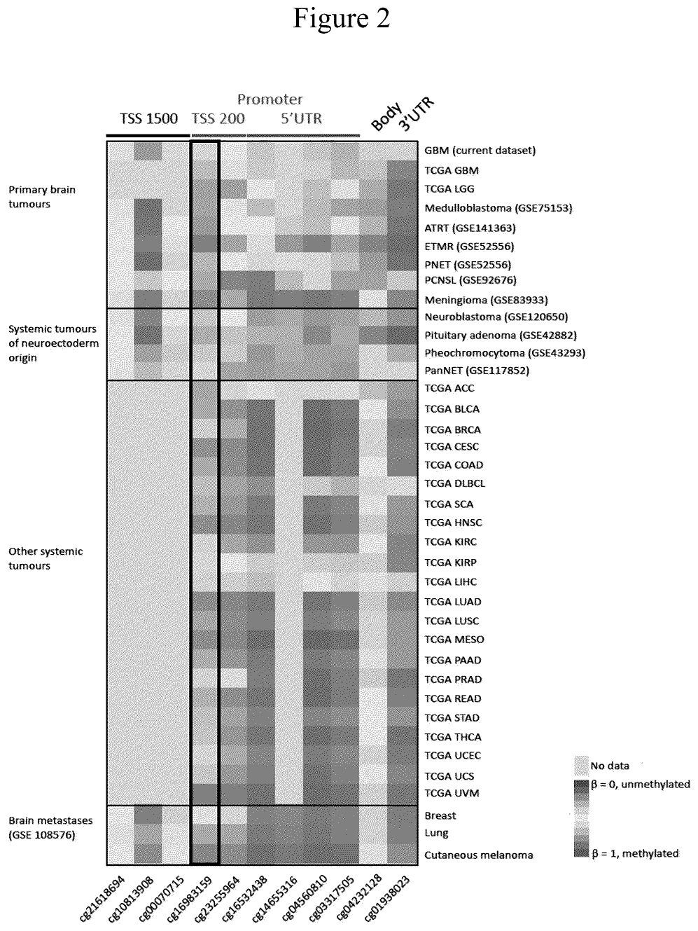

Similar to the case in glioblastoma, other brain cancers generally exhibit DNA hypermethylation at the cg16983159 STING promoter site (FIG. 2). In contrast, using TCGA data, we find that most cancers arising outside the nervous system are hypomethylated at this CpG site. However, there are a number of exceptions to this paradigm. Two intracranial tumor types tested exhibit STING promoter hypomethylation, rather than hypermethylation: primary CNS lymphoma (PCNSL) and meningioma. Interestingly, PCNSL is not derived from neuroectoderm tissue. It was therefore wondered if cancers of neuroectoderm origin tend to be methylated at cg16983159, while those that are not derived from the neuroectoderm tend to be unmethylated at this site. Tumors were analyzed that arise outside the nervous system but that are of neuroectodermal embryologic origin: neuroblastoma, pancreatic neuroendocrine tumor, pheochromocytoma, and pituitary adenoma. Indeed, like brain cancers, these tumors are on average methylated at the STING promoter cg16983159 site. However, melanoma (TCGA code UVM) and small cell lung cancer (TCGA code LUSC) are also derived from neuroectoderm cells but are not methylated at this site.

A few systemic cancers analyzed from TCGA are hypermethylated at the STING promoter but are not clearly derived from neuroectodermal tissue. The methylation status of the STING gene and STING mRNA expression level were queried in normal tissue samples to determine if hypermethylation is acquired by the cancer or if, like in the brain, their tissue-of-origin is already hypermethylated. In all cases except for prostate cancer, the normal tissue-of-origin also shows STING promoter cg16983159 hypermethylation and low STING mRNA expression.

Finally, lung, breast, and melanoma brain metastases STING methylation data was analyzed using the dataset of Orozco et al. (GEO accession GSE108576). When comparing these data with TCGA data, we found that methylation across the STING gene is very similar between brain metastases and their corresponding primary tumor sites (FIG. 2). Thus, STING appears to maintain its methylation program upon brain metastases for these tumor types.

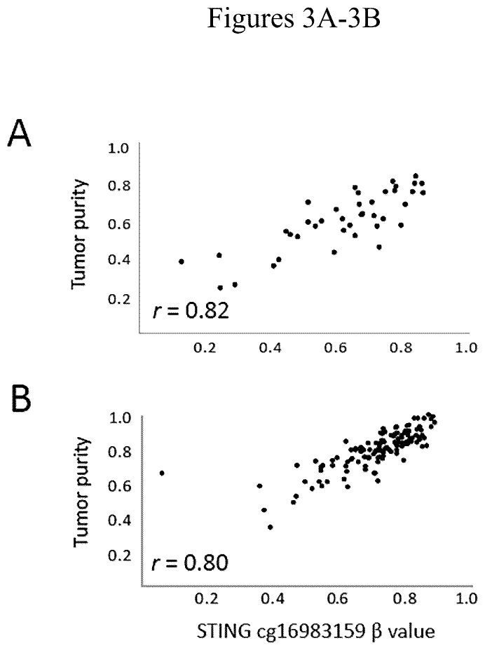

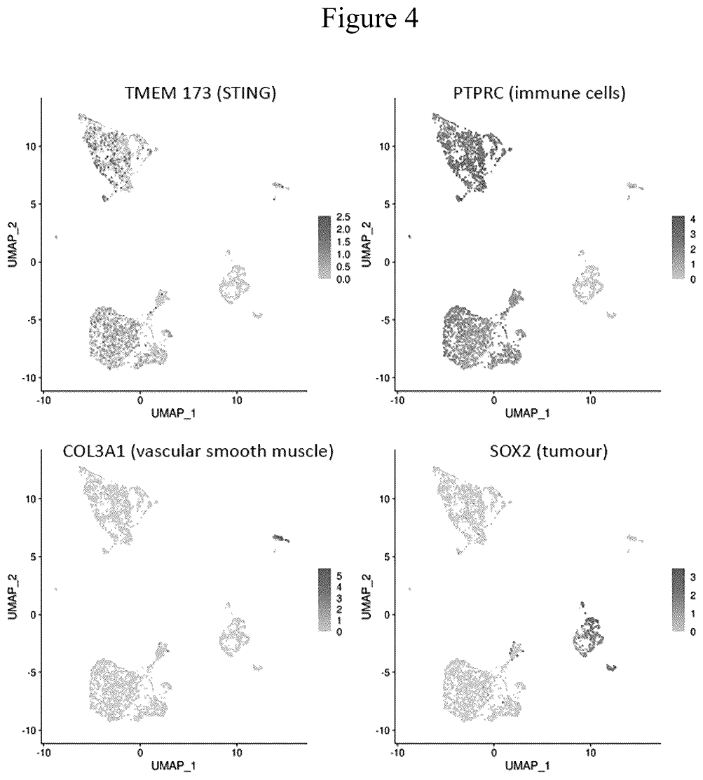

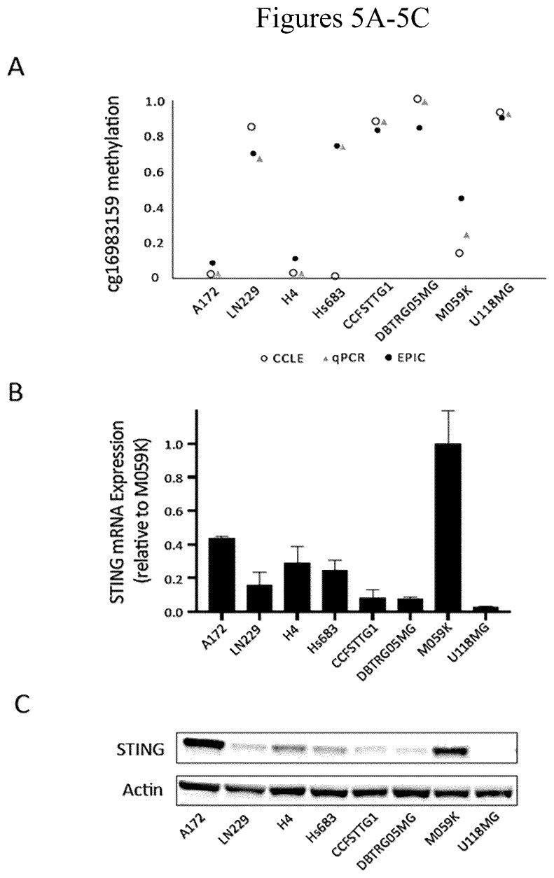

STING is expressed in immune cells but not tumor cells. While the STING promoter cg16983159 site is generally highly methylated in glioblastoma, a few samples exhibit lower methylation values (FIG. 1A). To determine whether hypomethylation in bulk patient samples represents lower methylation in the tumor itself or infiltration by immune cells (which are hypomethylated at this site), methylation with tumor purity was compared using both our current dataset and TCGA data calculated by Aran et al. (FIG. 3). It was found that methylation correlates very well with tumor purity: samples with low methylation values correspond to low tumor purity while highly pure samples are consistently hypermethylated. It was concluded that low methylation in bulk GBM tumor samples reflects contributions from immune cells. To directly examine STING expression in glioblastoma and its microenvironment, single-cell RNA (scRNA) sequencing experiments were performed on tissue from patients with GBM. It was found that STING mRNA expression is suppressed in tumor cells. In contrast, STING is expressed in immune cells and vascular smooth muscle cells (FIG. 4).

Functional data: STING expression is silenced in cell lines with cg16983159 hypermethylation: The above results demonstrate that in patient GBM tumor cells, STING is not expressed and that cg16983159 is always methylated. On the other hand, immune cells express STING and are unmethylated at cg16983159. Therefore, the strong inverse correlations between methylation and gene expression observed in bulk tumor data (FIG. 1B, FIG. 10) reflect the degree of immune infiltration rather than correlations from the tumor cells themselves. Thus, confirmation of the relationship between STING cg16983159 methylation and gene expression using in vitro glioma models were sought since these are free from the confounding effects of the tumor microenvironment. Eight glioma cell lines were chosen to represent a range STING promoter methylation values per the Cancer Cell Line Encyclopedia (CCLE). A qPCR-based assay was designed to determine the methylation of this specific CpG site. cg16983159 methylation was quantified using both this site-specific assay and Illumina 850 k EPIC arrays and these two methods were found to agree very well with each other (FIG. 5A, Pearson's r=0.98). Three cell lines showed low methylation (A172, H4, M059K) while the other lines showed moderate to high STING methylation (FIG. 5A).

Gene expression was compared with cg16983159 methylation in these glioma cell lines. Cell lines with high STING promoter methylation had low STING mRNA and protein expression (FIG. 5B-C). Of the 3 cell lines with low STING methylation, 2 (A172, M059K) clearly demonstrated high mRNA and protein expression. However, H4, which exhibited low promoter methylation showed low mRNA and protein expression. These data suggest that (1): hypermethylation at this CpG site is closely related to suppression of STING mRNA and protein expression and (2) when this site is unmethylated, epigenetic suppression is released, but that STING expression may still be regulated via other mechanisms.

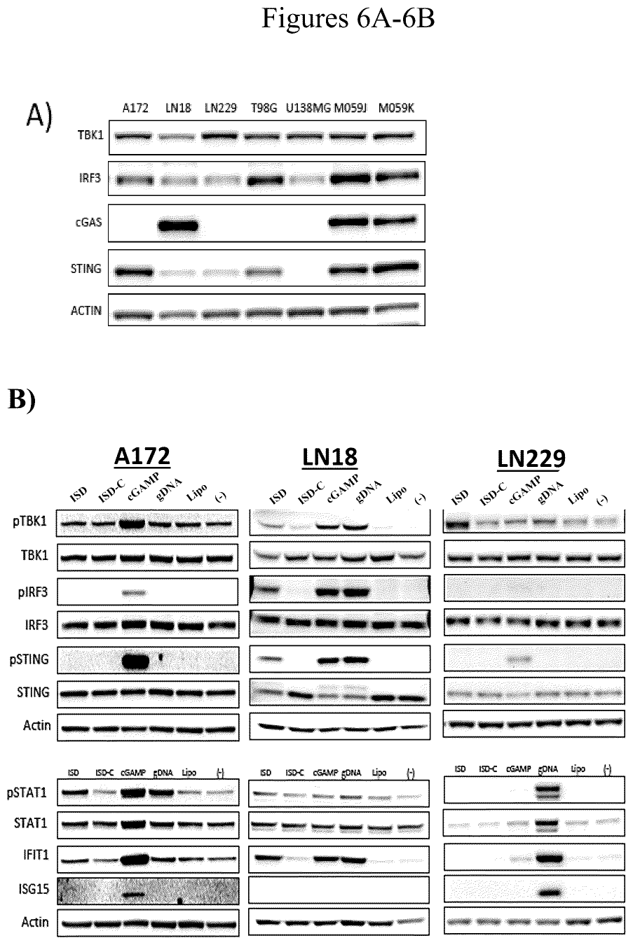

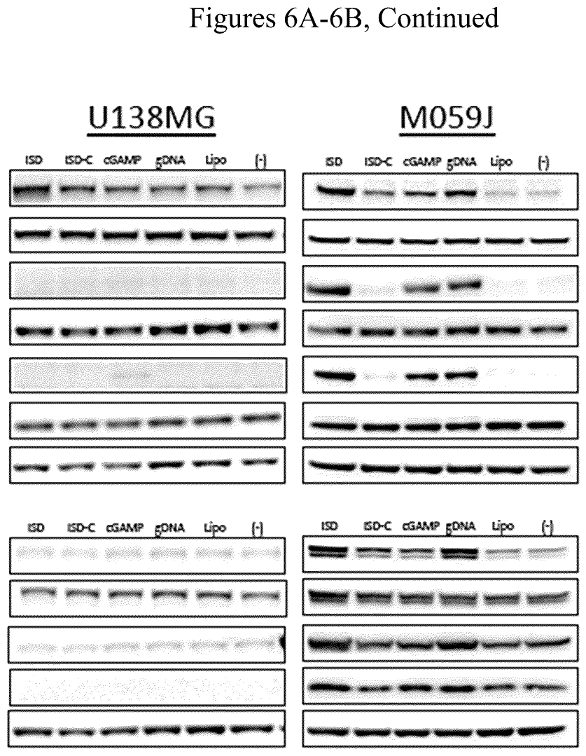

The cGAS STING DNA innate sensing pathway is variable and generally dysfunctional in GBM cell lines and does not recapitulate human tumors. The cGAS/STING DNA sensing pathway was investigated in greater detail using GBM cell lines. Baseline expression of key pathway mediators was characterized and then the response of downstream effectors to treatment with DNA agonists was measured (FIG. 6). Cells were treated with ISD (cGAS agonist), 2′3′-cGAMP (STING agonist), and self-gDNA, and protein harvested at 4 and 24 h. The immediate downstream response to these agonists was evaluated by measuring the expression of phosphorylated TBK1, IRF3, and STING (FIG. 6B, upper panel). All cell lines expressed TBK1 and IRF3. However, expression of cGAS and STING was highly variable amongst the different cell lines. Predictably, cell lines lacking expression of cGAS showed no response to cGAS agonist ISD. Response to STING agonist cGAMP was variable: The A172 cell line with high baseline STING expression resulted in a strong downstream response, while the U138MG cell line with no STING expression failed to activate pTBK1 or pIRF3. Only the cell lines with both cGAS and STING expressed at baseline (M059J, LN18) demonstrated intact pTBK1 and pIRF3 expression in response to all DNA agonists tested.

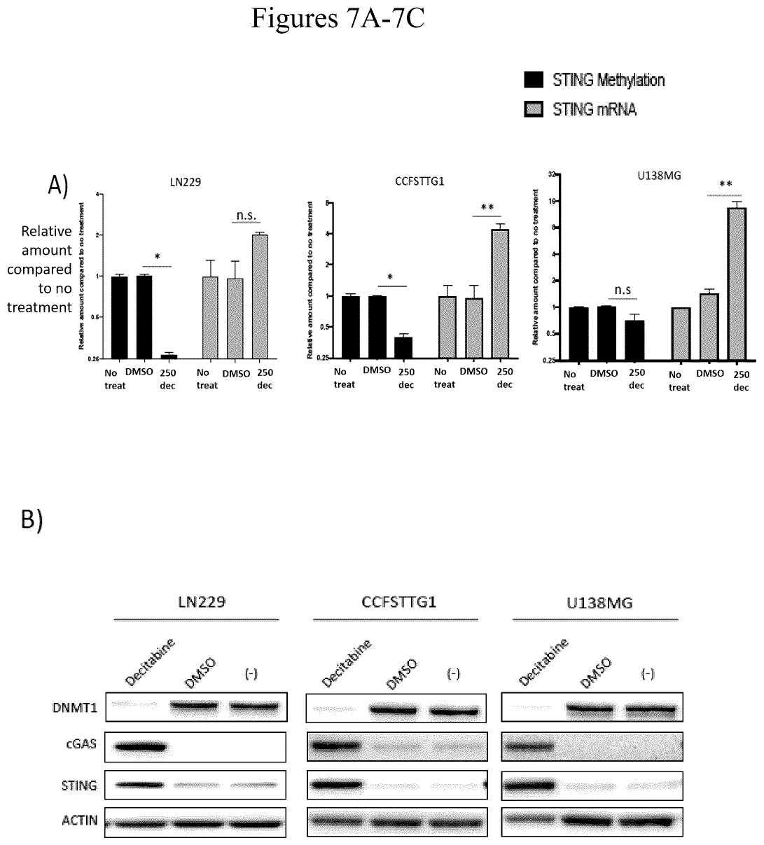

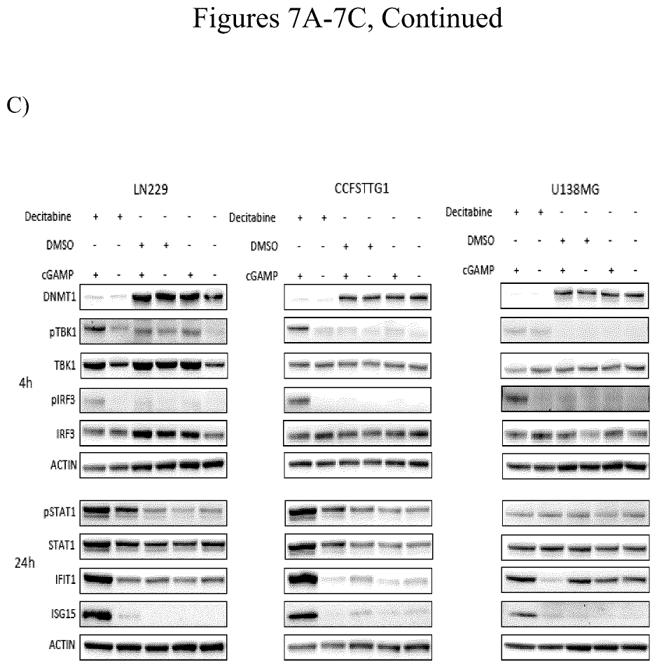

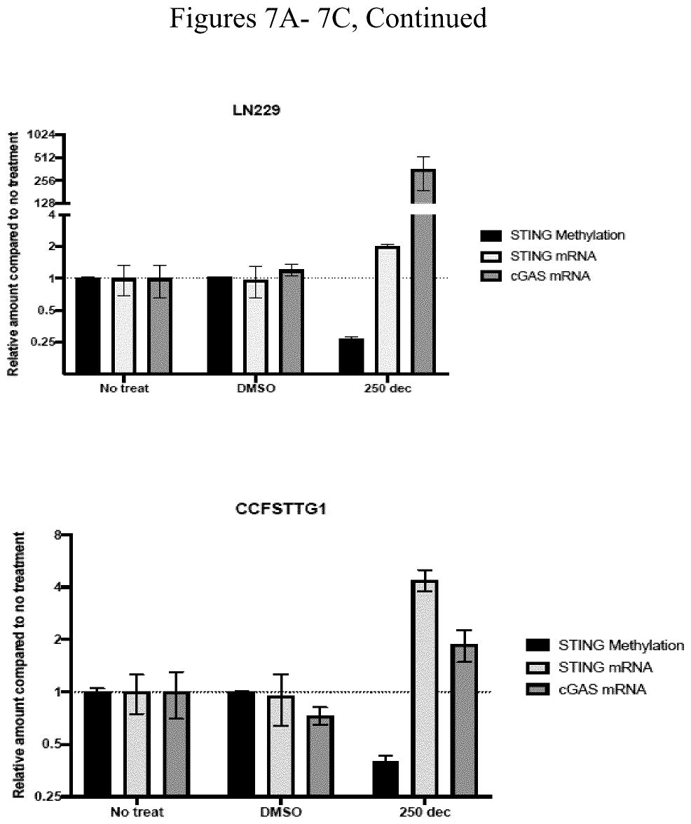

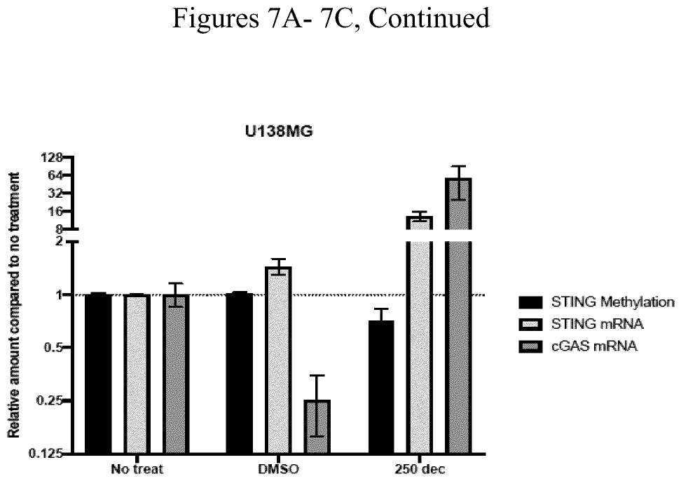

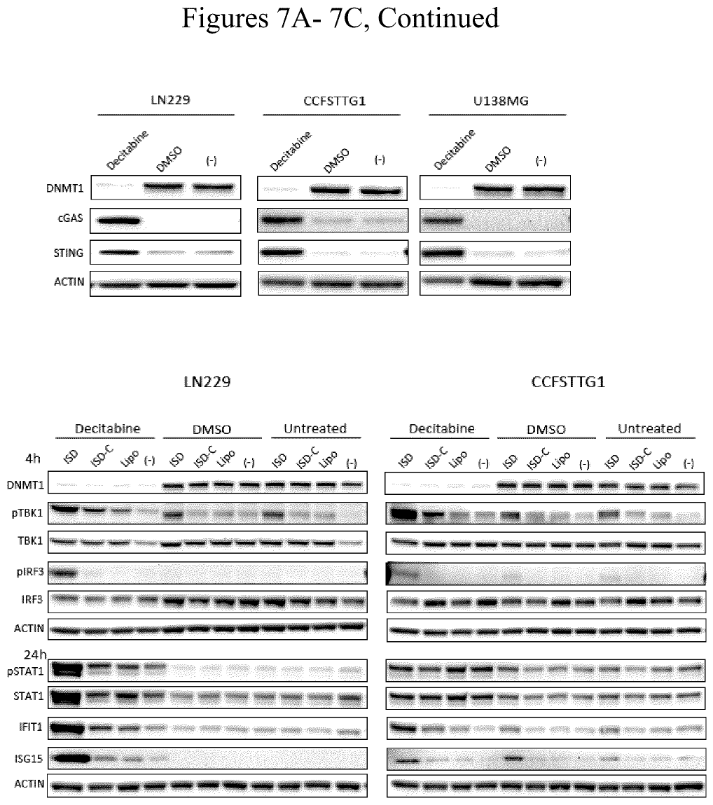

Since cGAS/STING signaling ultimately activates an interferon response, the expression of markers pSTAT1, IFIT1, and ISG15 was measured in response to DNA agonists (FIG. 6B, lower panel). It was found that only the M059J cell line activates all of these markers in response to all stimuli tested. Collectively, these experiments show that in general GBM cell lines do not recapitulate the pattern of cGAS/STING expression we observed in patient samples (where, for example, STING is universally not expressed). Additionally, they suggest that baseline STING and cGAS expression are necessary but not sufficient alone to activate pTBK1 and pIRF3 for downstream interferon responses in GBM cell lines. Decitabine demethylates the STING promoter and upregulates RNA and protein expression in glioblastoma cell lines. Despite the limitations in GBM cell lines they can be interrogated to examine STING expression and signaling. Therefore, it was evaluated whether cg16983159 could be demethylated and if this would rescue STING expression. Three cell lines were chosen that best represented human tumor cells with baseline high STING promoter methylation and low RNA/protein expression. Cells were treated with decitabine (5Aza-CdR, Dacogen®), a DNA methyltransferase 1 (DNMT1) inhibitor that is approved for the treatment of myelodysplastic syndrome and acute myeloid leukemia. Cells were treated with decitabine for 5 days, harvested 24 h later, and then tested for STING cg16983159 methylation, RNA and protein expression. Decitabine treatment resulted in significant suppression of DNMT1, decreased cg16983159 methylation, and increased STING mRNA and protein expression in all 3 cell lines treated (FIG. 7A-B).

Since decitabine treatment induced STING expression, it was next tested whether cell lines previously unresponsive to the STING agonist cGAMP were now able to induce the pathway when pretreated with decitabine. Indeed, pretreatment with decitabine for 5 days followed by cGAMP treatment resulted in reactivation or enhancement of the pathway in all three cell lines, as measured by pTBK1 and pIRF3 expression (FIG. 7C). Markers of downstream IFN activation (pSTAT1, IFIT1, and ISG15) are also induced in the decitabine- and cGAMP-treated cells.

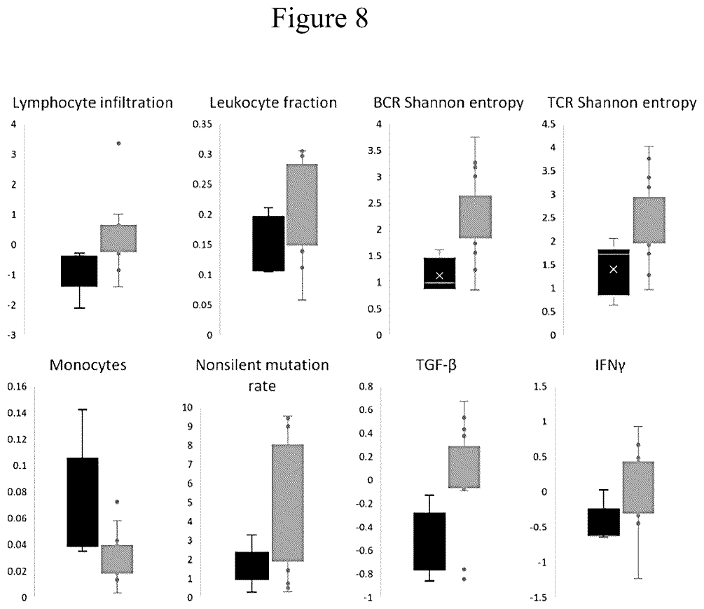

Cancers with STING promoter hypermethylation are immunologically cold. Given the importance of STING signaling in innate immunity, the broader immunologic status of cancers where this pathway is silenced by STING promoter methylation were queried. Using calculations from TCGA data that were previously performed, it was analyzed to see if there were any significant differences in immune marker levels between cancers with mean cg16983159 methylation values >0.6 and those <0.3 (FIG. 8). It was found that cancers with high STING promoter methylation show low levels of immune markers including measures of leukocyte infiltration, B- and T-cell repertoire, neoantigen load, mutational rate, TGF-β, and IFNγ. Correspondingly, cancers with low STING promoter methylation values display comparatively higher immune markers, with the notable exception of uveal melanoma (TCGA code UVM). It was concluded that cancers where STING is epigenetically silenced are overall immunologically cold.

### B. Discussion

The results above provide insights into the mechanisms of STING silencing in the GBM tumor microenvironment. Hypermethylation has been recognized as a common mechanism of STING silencing in cancer. In pan-cancer analysis of TCGA data, it was demonstrated that increased methylation of STING and cGAS are commonly seen in a subpopulation of tumors as compared to their corresponding normal tissues. A wide range of methylation values were observed in samples of high tumor purity. One interpretation is that hypermethylation develops in oncogenesis as a mechanism of STING epigenetic silencing in an otherwise epigenetically intact STING environment. The results find the situation in GBM to be quite different from this general cancer paradigm. Correlations between methylation and tumor purity show that the cg16983159 STING promoter site is exclusively hypermethylated in cancer cells, and that lower methylation values seen in bulk GBM tumor samples occur only when tumor purity is low and are due to contributions from infiltrating immune cells (FIG. 3). Single-cell RNA sequencing experiments confirm the lack of STING expression in individual tumor cells (FIG. 4). The observation that STING is also silenced in the normal brain implies that STING hypermethylation is not likely a consequence of gliomagenesis, but rather that GBM forms in the context of a normally STING-silent environment. STING hypermethylation appears to be maintained across multiple cerebral disease states, including neurodegenerative conditions such as Alzheimer and Parkinson diseases; neuroinflammatory conditions such as multiple sclerosis; and psychiatric disease such as bipolar disorder and schizophrenia.

Interestingly, other primary brain tumors that presumably derive from neural tissues also display STING promoter hypermethylation, such as low-grade gliomas and medulloblastomas. The fact that extracranial tumors of neuroectodermal origin also exhibit STING promoter hypermethylation raises the possibility that epigenetic silencing occurs early in the development of these cells and that this hypermethylation is maintained throughout development and oncogenesis. The single cell RNA expression analysis shown herein demonstrating that microglia can express STING is consistent with this notion as microglia are myeloid cells of mesenchymal origin. In contrast, meningiomas are not hypermethylated in bulk patient samples even though the meninges are derived from the neuroectoderm. It is important to note that although meningiomas are thought to originate from neuroectoderm-derived arachnoidal cells, the meningioma cell-of-origin has not been definitively established. Additionally, the meningioma immune microenvironment may be quite different from that of gliomas and it is not clear if lack of STING hypermethylation could be due in part to immune infiltration.

The pan-cancer analysis of methylation at cg16983159 revealed that in addition to cancers of neuroectodermal origin, a minority of extracranial cancers also exhibit hypermethylation at this site. In nearly all of these cases, the normal tissue-of-origin is also hypermethylated. The main exception is prostate adenocarcinoma, which displays hypermethylation while normal prostate does not. Thus, STING promoter methylation could be one avenue of immunosuppression in the development of prostate cancer. A few other cancer types, such as renal papillary carcinoma, are not hypermethylated although they arise in STING hypermethylated tissues. These observations suggest that the methylation status of the STING promoter can change upon oncogenesis in certain tissue types. When analyzing individual patient methylomes, we found that systemic cancers where the STING cg16983159 is on average unmethylated in the overall population still include a subset of patients where this site is methylated (data not shown). These results agree with those previously reported by Konno et al in their pan-cancer methylation analysis. The inventors hypothesized that such tumors, like glioblastoma, exhibit STING epigenetic silencing. The inventors term these tumors “STING-silenced tumors” and hypothesize that they will behave similarly to glioblastoma and will not respond well to standard anti-cancer therapy.

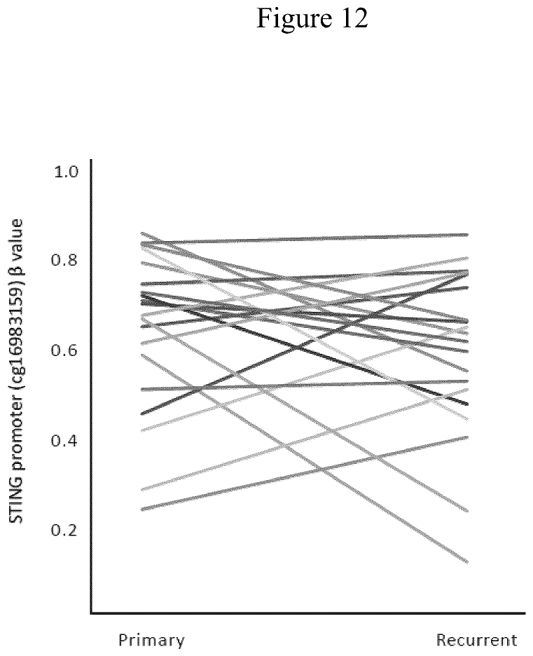

Whether STING methylation in cancer cells has a propensity to change, either as a result of selective pressures or simply neutral evolution, remains an open question. Analysis of the data of lung, breast, and melanoma brain metastases show that methylation values across the STING gene are very similar in the brain as compared to their corresponding primary tumors, despite significant changes in their microenvironments (FIG. 6). On the other hand, the data from pre- and post-treatment GBM patient samples indicate that bulk methylation values are different at disease progression, though there is no apparent trend towards more or less hypermethylation (FIG. 12). Since these methylation values correlate strongly with tumor purity, this likely implies changes in the degree of immune infiltration rather than changes in methylation of the tumor cell itself.

The relationship between STING methylation and overall tumor immunogenicity is underscored by the observation that STING-methylated tumors are associated with immunologically “cold” cancer types. If this relationship proves causal, then agents that demethylate, either in a nonspecific manner or potentially in a site-directed manner, may relieve STING suppression in GBM and surrounding cells. This could allow cancer cells to signal their pathogenic status and attract host immune cells. An important caveat is that a significant fraction of GBMs contain large-scale homozygous deletions of chromosome 9p that includes CDKN2A and interferon-1, calling into question whether rescued STING expression would be sufficient for triggering a complete interferon response in these tumors. On the other hand, intact STING signaling within tumor cells may not be required for extrinsically activating STING in infiltrating immune cells. Results showed that cytosolic DNA in STING-negative cells can trigger STING activation in co-cultured antigen presenting cells with an intact STING pathway. Whether intratumoral intrinsic STING signaling is still necessary to alert APCs to the tumor cell, and the precise crosstalk between intrinsic tumor DNA sensing and extrinsic STING signaling remains to be elucidated. Finally, STING agonists have attracted attention as potential anticancer therapeutics and may be able to stimulate a STING-dependent immune response in GBM. Strategies that activate STING signaling may act synergistically with other immunostimulatory therapies, such as PD-1 blockade in an otherwise immunologically cold environment.

In summary, the present study adds to the growing body of work demonstrating STING dysregulation in cancer. Unusually amongst normal human tissues, STING is epigenetically silenced by promoter methylation in the brain, which may contribute to its unique immunosuppressive environment in which gliomas and other primary brain tumors develop.

### C. Methods

Cell culture: Cell lines were obtained from the Duke Cell Culture Facility and were grown in media according to ATCC protocols. Cell lines were authenticated with STR profiling by the Duke CCF and were routinely tested for mycoplasma contamination.

Glioblastoma patient samples: DNA was extracted from glioblastoma patients in the The Preston Robert Tisch Brain Tumor Center BioRepository (accredited by the College of American Pathologists) with approval from the Institutional Review Board. Tissue sections were reviewed by board-certified neuropathologists to confirm histopathological diagnosis of glioblastoma. Samples with ≥60% tumor cellularity by hematoxylin and eosin (H&E) staining were selected.

Nucleic Acid Extraction: DNA and RNA were extracted from either cells or snap-frozen tumor tissue using Qiagen AllPrep DNA/RNA mini kit following the manufacturer's protocols.

Methylation arrays: Genomic DNA was provided to the Duke Molecular Genomics Core who performed bisulfite conversion with the Zymo EZ methylation kit followed by Illumina MethylationEPIC Array screening using the manufacturer's protocols. Arrays were read on an Illumina iScan instrument and the resulting data processed using the Minfi package in R to calculate methylation beta values, where beta=0 repressed a completely unmethylated CpG site and beta=1 represents a fully methylated site.

Methylation datasets: For the glioblastoma patient sample analyses, two sets of methylation array data were used. In the first dataset we used Illumina EPIC array data on extracted samples from Duke as described above. The second methylation dataset included paired sets of pre- and post-treatment samples from 21 glioblastoma patients obtained from the Royal Melbourne Hospital (RMH) and University of Melbourne, Department of Surgery Brain Tumor Bank as previously described (Muscat). Briefly, in this second dataset, DNA was extracted from Formalin-fixed paraffin-embedded (FFPE) tumor samples using ReliaPrep™ FFPE gDNA Miniprep System (Promega), and bisulfite converted using MethyEasy™ Bisulphite Kit (Genetic signatures) by The Centre for Applied Genomics, The Hospital for Sick Children (Toronto, Canada) per manufacturer's instructions. The Illumina 450K bead array was then performed, and methylation beta values calculated. These paired methylation data were previously deposited in the Gene Expression Omnibus (GEO) under accession number GSE85087. Methylation beta values from TCGA were downloaded using the Wanderer portal (maplab.imppc.org/wanderer/).

Other methylation datasets from patients with cancers not in the TCGA, brain metastases, non-cancerous neurologic disease states, and normal brain samples were downloaded from GEO with the accession numbers provided in the manuscript and figures.

Methylation Data Analysis: Methylation beta values for each of the TMEM173 gene CpG probes (11 for the 450 k arrays and 16 probes for the EPIC array) were extracted from data files using custom scripts.

Tumor Purity Calculations: Tumor purity for our glioblastoma patient samples was estimated using Leukocytes unmethylation to infer tumor purity (LUMP) analysis as previously described (Aran). LUMP estimations are based on the average methylation levels of 39 of the informative CpG sites. In the Melbourne cohort of 21 paired patients, probes cg00240653, cg02997560, cg03436397, cg26427109 and cg05199874 were excluded from the analysis as the detection p-value for these was not below a threshold (T) of 0.05. For TCGA data, the tumor purity calculations performed by Aran et al. were used.

Decitabine Treatment: Decitabine was dissolved in DMSO to produce a stock concentration of 10 mM. This stock concentration was diluted directly before each treatment at a 1:100 ratio with 1×PBS to create the working solution with a concentration of 100 PM.

Cells were seeded at density of 30,000 cells/well in 6-well plates. Each cell line was seeded in 2 mL of the same media in which they were cultured (cells were cultured in media according to ATCC). The following day (Day 0), a volume of decitabine working solution was added directly to the wells to reach the desired concentration. In the DMSO control wells, DMSO was diluted 1:100 in PBS, with the same amount of DMSO:PBS solution added as that of the decitabine condition. On days 1-5, the media was refreshed, such that the media being added to the wells contained the respective decitabine/DMSO concentration and were prepared immediately before their addition. The cells were harvested for protein on day 6.

Protocol for mRNA Expression: RNA expression was measured by converting 250 ng to lug of RNA to cDNA with the iScript cDNA conversion kit (BioRad Cat #1708891) in 20 μL total. Thermal cycling conditions as follows:

| step | Temperature, ° C. | Time, min |
|---|---|---|
| Prime | 25 | 5 |
| RT | 46 | 20 |
| RT inactivation | 95 | 1 |
| Hold | 4 | ∞ |

The cDNA reaction was diluted to a final concentration of 2-10 ng/μL and combined with the following in 10 μL total.

| # rxns | 1 | |
|---|---|---|
| 20× PrimePCR | 0.5 | uL |
| 2× SsoAdvanced (BioRad cat# 1725271) | 5 | uL |
| water | 2.5 | uL |
| cDNA (variable up to 20 ng) | 2 | uL |

20× PrimePCR Primers targeting B-actin (BioRad #qHsa CED0036269), and TMEM173 (BioRad #qHsa CID00010565), were used. Fold change (2\hat{ }(−Delta Delta Ct) was calculated using B-actin as the housekeeping gene.

Single-cell RNA-Sequencing Protocol (scRNA-Seq): Tumor samples were enzymatically dissociated using Collagenase A (Sigma), and the single-cell suspension was stained with live-dead (BioLegend) stain to exclude dead cells. The cell suspension was next treated with Fc receptor block followed by antibodies specific for CD45, CD3, CD64, CD163, EGFRwt, and Podoplanin (PDPN). Stained cells were subjected to a 3-way sorting for tumor (CD45, EGFRwt+ PDPN+), myeloid (CD45+CD64+CD163+), and T Cells (CD45+CD3+). Post-sorting enriched tumor, myeloid, and T cells were pooled and processed for droplet-based RNA sequencing following the manufacturer's protocol (10× Genomics). cDNA isolation and gene expression library preparation were completed as per the manufacturer's protocol using Chromium Single Cell 3′ Reagent Kit (10× Genomics). The gene expression libraries were sequenced on the Illumina NovoSeq platform with 150 bp paired-end read configuration. Raw fastq files were analyzed using Cellranger 3.1 software. Reads were mapped to the pre-built GRCh38-3.0.0 reference from 10×'s website. Read lengths were hard-trimmed to 10×'s recommendations for 3′ v3 chemistry.

Single-cellRRNA-Sequencing Analysis: Seurat version 3 was used for downstream analysis. The output of cellranger count from each sample was used as separate inputs into a workflow based on the Seurat data analysis package. First, doublets were detected and removed using doubletCells, part of the scran package from the open-source Bioconductor project (Huber et al., 2015). Next, outliers were calculated based on the following three metrics: mitochondrial percentage, number of features, and number of UML. Cells identified as outliers were excluded. Then, the Seurat method SCTransform was applied. Variance due to mitochondrial content, number of features, number of genes, and the difference between the G2M and S phase scores was regressed out. Next, PCA was performed on the scaled data. Using the first 25 PCA dimensions, FindNeighbors and FindClusters were performed to identify clusters. Clusters were visualized in a Uniform Manifold Approximation and Projection embedding (UMAP) plot. Significantly expressed genes in each cluster compared to the rest of the dataset were identified using the FindAllMarkers method from Seurat. Expression of TMEM173, PTPRC (immune cells), COL3A1 (endothelial cells), and SOX2 (tumor cells) genes were visualized by feature plots.

STING Methylation qPCR Methods: Bisulfite conversion of 25 ng to lug gDNA was done using the EpiTect Bisulfite Kit (Qiagen 59104) following manufactures protocol. STING methylation qPCR was performed on 1-10 ng of bisulfite converted DNA in 1×PCR Buffer with 0.5 μM of each TMEM173 forward 5′-TAGAGGAATGGGGGTTTGGT-3′ (SEQ ID NO:1) and reverse 5′-TCCTACCTAATATCATCCCCACAA-3′ (SEQ ID NO: 2) primers (IDT), 0.15 μM of each methylated FAM-TGTAGGAAATGGTTAcGTT-MGB-NFQ (SEQ ID NO: 3) and unmethylated VIC-TGTAGGAAATGGTTAtGTTT-MGB-NFQ probes (SEQ ID NO: 4), 0.75 mM MgCl₂, 0.2 mM dNTPs, and 0.75U HotStar Taq (Qiagen 203205). Percent methylation was calculated as 1/(1+1/(2\hat{ }-ΔCt))×100 with ΔCt=FAM −VIC. Cycling conditions were as follows:

| | Time | Temperature (C.) |
|---|---|---|
| Initial activation | 15 min | 95 |
| Denaturation | 30 sec | 94 |
| Annealing | 60 sec | 62 |
| Extension | 60 sec | 72 |

Western Blots: Cells were washed 2× with ice cold 1×PBS and lysed on ice with RIPA lysis buffer, containing protease/phosphatase inhibitors, and benzonase. Lysates were spun down for 15 min 14,000×g at 4 degrees C. Protein was denatured, ran on 4-12% NuPage Bis-Tris gels in 1×MOPS buffer, and transferred to PVDF membrane. The membrane was blocked in 5% Blotting grade blocker (BioRad 170-6404) dissolved in 1×TBS 0.1% TWEEN. Primary antibody was added according to manufacturer's protocol and incubated overnight at 4° C. Membranes were then incubated with secondary HRP antibody and screened with Supersignal Pico ECL. A ChemiDoc MP (BioRad) and an iBright 1500FL imager (ThermoFisher) were used to detect the ECL signal.

| Antibody | Cell Signaling Number |
|---|---|
| Secondary- Anti-rabbit HRP | CST #7074 |
| Secondary - Anti-mouse HRP | CST #7076 |
| Beat-Actin HRP conjugate | CST #5125 |
| cGAS (human specific) | CST #15102 |
| DNMT1 | CST #5032 |
| IFIT1 (human specific) | CST #14769 |
| ISG15 | CST #2743 |
| IRF-3 | CST #4302 |
| IRF-3 (human & mouse) | Biolegend #655702 |
| p-IRF3 | CST #4947 |
| p-STAT1 | CSt #9167 |
| STAT1 | CST #9172 |
| p-STING human | CST #19781 |
| STING | CST #13647 |
| p-TBK1 | CST #5483 |
| TBK1 | CST #3504 |

### Example 2—STING Silencing in Gliomas and Other Cancers is Mediated by Site Specific and Developmental Tissue-of-Origin Promoter Hypermethylation and can be Rescued by Methyltransferase Inhibition

The stimulator of interferon genes (STING) is a critical signaling transducer of cytosolic DNA sensing, eliciting IRF3- and NFkB-dependent transcription of type I IFNs and pro-inflammatory cytokines. The importance of tumor-localized STING activation in priming endogenous antitumor immunity is well established. A pan-cancer analysis showed that STING signaling is frequently disrupted through loss-of-function mutations or hypermethylation of the STING or cGAS promoters (Konno et al., 2018). Here we investigate STING signaling in glioblastoma (GBM) and normal brain and find that STING expression is suppressed in both normal brain and glioma cells but not tumor-associated immune cells or stroma. We identify a CpG site that is methylated in normal brain, gliomas, and other neuroectoderm-derived cancers, but not in most extracranial cancers. We demonstrate that STING expression is rescued by decitabine, a DNA methyltransferase inhibitor (DNMTi) that is used to treat myelodysplastic syndrome and acute myeloid leukemia. Our work raises the potential of DNMT is to reconstitute STING signaling in GBM tumor cells.

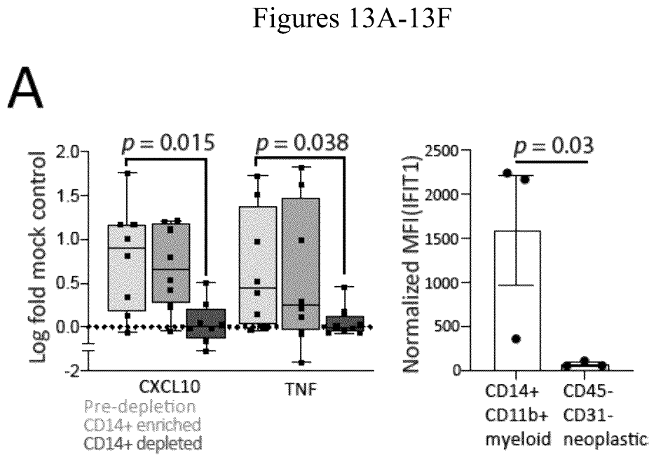

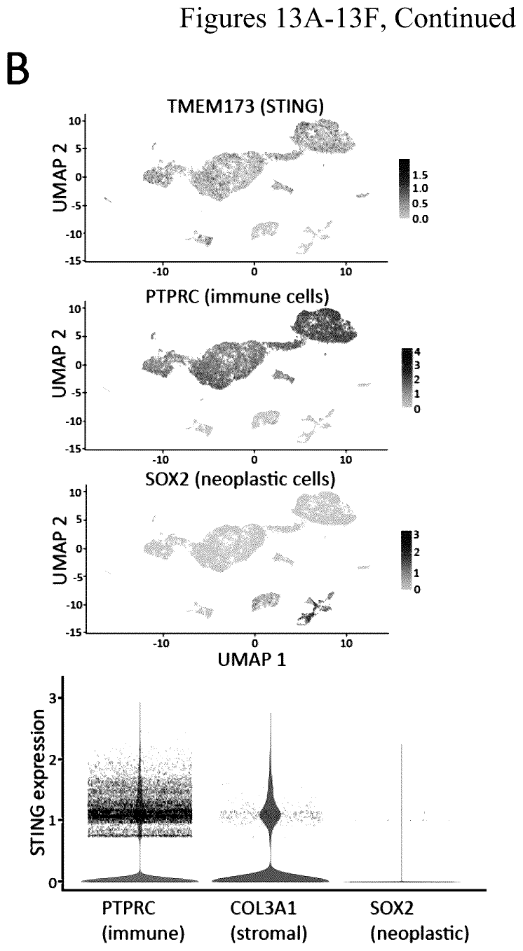

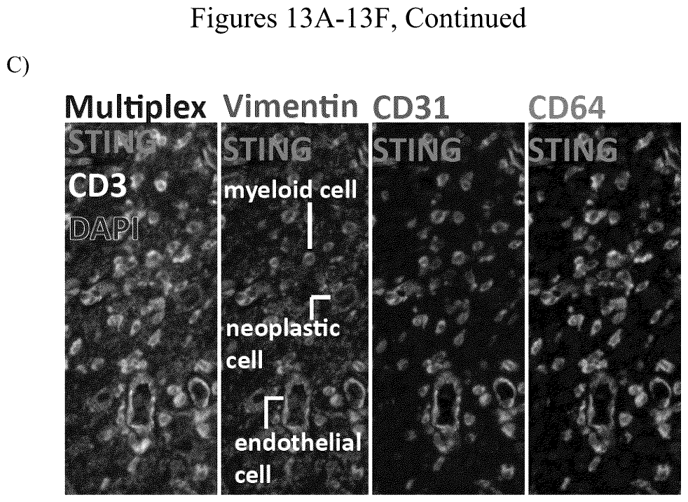

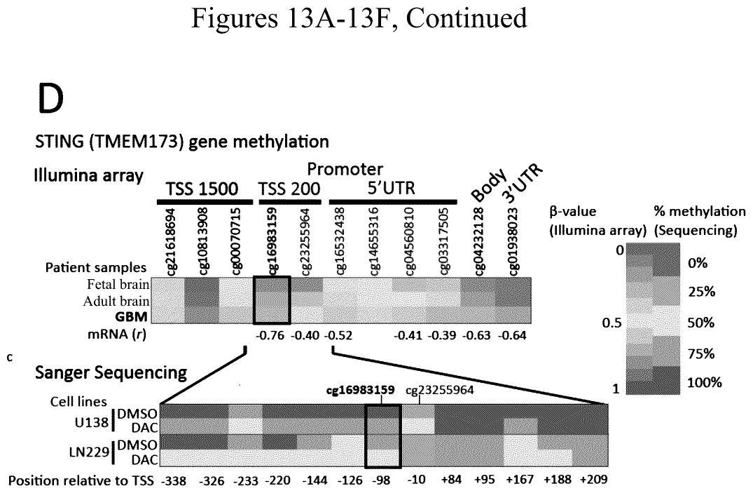

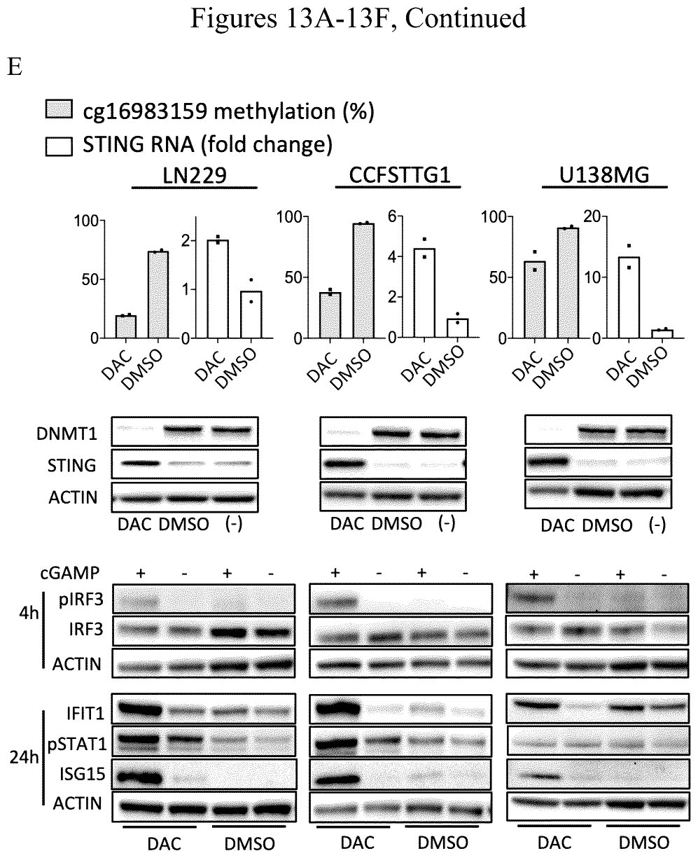

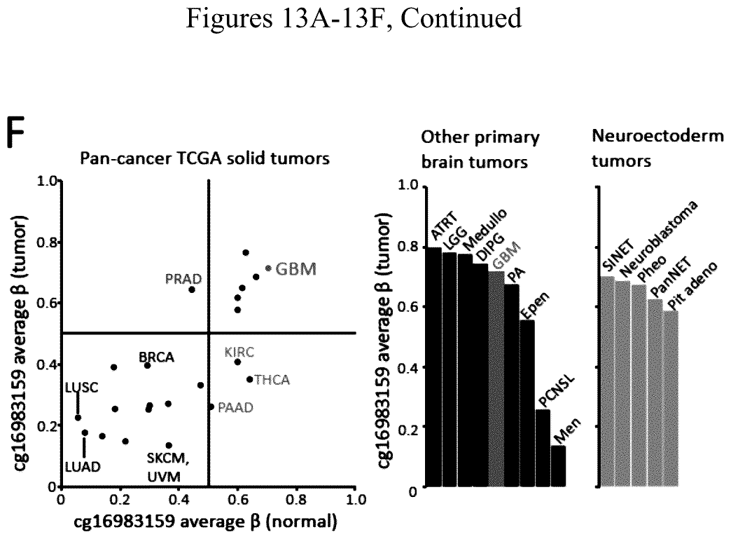

We examined whether the cGAS/STING pathway is disrupted in GBM, as has been demonstrated in other cancers (Xia et al., 2016a, 2016b). We recently reported that the STING agonist 2′3′-cGAMP (cGAMP) induced type I IFNs and CXCL10 in ex vivo GBM tissue slice cultures that retained the native tumor microenvironment (Brown et al., 2021). Here we separated GBM cell suspensions by surface CD14 expression, a specific marker of tumor-associated myeloid cells (TAMC). cGAMP-mediated CXCL10 and TNF induction occurred only in CD14+ containing glioma cell suspensions, and the interferon-stimulated gene IFIT1 was induced selectively in TAMC after ex vivo GBM slice culture treatment (FIG. 13A), indicating that STING signaling is absent in the neoplastic, but not myeloid compartment of human GBM samples.

We investigated baseline STING expression using single-cell RNA-sequencing profiles in neoplastic, immune, and stromal cells from newly diagnosed GBM patient samples (FIG. 13B). Single-cell clustering analysis identified three major clusters: neoplastic (SOX2), immune (PTPRC), and vascular smooth muscle cells (COL3A1). Detectable STING mRNA was observed only in immune and stromal cells, but not in neoplastic cells. Multiplex immunofluorescence analysis of GBM tumor sections was performed to colocalize STING expression with CD3 (T-cells), CD64 (macrophage/microglia), CD31 (endothelial cells), and vimentin (mesenchyme) (FIG. 13C). STING protein expression was restricted to myeloid and endothelial/stromal cells and was absent in GBM tumor cells and T cells (FIG. 13C).

Despite absent STING expression and signaling in GBM cells, STING gene mutations are rare, with only one out of 378 samples in TCGA GBM PanCancer Atlas exhibiting a missense mutation. We hypothesized that epigenetic silencing of STING could account for its reduced expression. We performed Illumina methylation arrays on 64 GBM patient samples, analyzed the methylation values across the STING gene, and compared them with those from normal fetal and adult brain datasets (Gasparoni et al., 2018). The CpG site cg16983159, located in the STING promoter (Wang et al., 2016), is consistently hypermethylated (FIG. 13D). TCGA GBM Illumina methylation array data are limited to 7 probes across the STING gene. Each of these probes demonstrates negative correlations with mRNA expression, with the strongest correlation being with probe cg16983159 (r=−0.76, FIG. 13D). These data suggest that cg16983159 methylation suppresses STING mRNA expression. The presence of cg16983159 hypermethylation in normal adult and fetal brains suggests that STING promoter hypermethylation is not a consequence of gliomagenesis, but rather that GBM forms in the context of a normally STING-silent environment that is conserved through brain development and subsequent tumorigenesis.

Since the Illumina methylation array probes only 11 CpG sites distributed across the STING gene, we sequenced two bisulfite-treated glioma cell lines to evaluate the methylation state of 13 CpG sites in a ˜400 nucleotide window centered on cg16983159 (See SEQ ID NO: 13 and 14). We chose cell lines that, like patient-derived samples, exhibit baseline high STING cg16981359 methylation and low STING expression. Moderate (LN229: 50-75%) to high (U138: 75-100%) methylation was seen at most CpG sites in this region. We hypothesized that STING expression and signaling are suppressed by methylation in the vicinity of cg16983159, which could be reversed by DNMT is. We treated cells with the DNMTi decitabine and found that cg16983159 and upstream CpG sites are demethylated, while downstream sites are not (FIG. 13D; site labeled as −98 of FIG. 13D is the cg16983159 site and is shown as position 279 of SEQ ID NO: 15; site labeled as −126 of FIG. 13D is at position 251 of SEQ ID NO: 15; site labeled as −144 of FIG. 13D is 233 of SEQ ID NO: 15; site labeled as −220 of FIG. 13D is at position 157 of SEQ ID NO: 15; site labeled as −233 of FIG. 13D is at position 144 of SEQ ID NO: 15; site labeled as −326 in FIG. 13D is at position 51 of SEQ ID NO: 15; site labeled as −338 of FIG. 13D is at position 39 of SEQ ID NO: 15). We additionally specifically measured STING cg16983159 methylation, RNA and protein expression. Decitabine treatment suppressed DNMT1, decreased cg16983159 methylation, and increased STING mRNA and protein expression (FIG. 13E). Moreover, cell lines previously unresponsive to the STING agonist cGAMP were able to activate innate immune and interferon-stimulated genes (e.g., p-IRF3, IFIT1, pSTAT1, and ISG15) after decitabine treatment.

Finally, we queried whether STING hypermethylation occurs in other tumor types. We plotted average cg16983159 methylation values for TCGA solid cancers versus non-cancer tissue (FIG. 13F). Most cancers arising outside the nervous system displayed cg16983159 hypomethylation (beta <0.5), as did their normal tissues-of-origin. Conversely, the few extracranial cancers that exhibited cg16983159 hypermethylation also demonstrated cg16983159 hypermethylation in their tissues-of-origin. Analysis of methylation datasets for other primary brain tumors from the Gene Expression Omnibus revealed cg16983159 hypermethylation, with two exceptions: primary CNS lymphoma and meningioma. Similar to primary brain cancers, neuroectoderm-derived tumors are also methylated at the STING promoter cg16983159 site (FIG. 13F). An exception to this finding was melanoma (TCGA codes UVM and SKCM), which is derived from neuroectoderm cells but is unmethylated at this site.

GBM carries frequent extrachromosomal and cytoplasmic DNA (Kim et al., 2020) that should invoke a cGAS/STING signal but yet has a “cold” micro-environment and is notoriously resistant to immunotherapy. Our work suggests that hypermethylation in the STING promoter mediates STING silencing in GBM and may contribute to its intrinsic immunosuppression. The utility of DNMTi to rescue STING signaling and induce sensitivity to STING agonists has been demonstrated in KRAS-LKB1-mutant lung cancer, where LKB1 loss results in hyperactivation of DNMT1 and STING promoter methylation (Kitajima et al., 2019). Here, we highlight STING epigenetic silencing as characteristic of both the normal brain and primary brain tumors. We propose that reconstituting endogenous STING signaling using DNMTi may be a promising approach for inducing immunotherapy sensitivity in GBM.

Methods Cell culture: All cell lines were obtained from the Duke Cell Culture Facility (CCF) and grown in media according to ATCC protocols at 37° C. and 5% CO2. Cell lines were authenticated with STR profiling by the Duke CCF and were routinely tested for mycoplasma contamination using the MycoAlert Plus kit (Lonza).

GBM patient samples: Samples used for DNA methylation, RNA expression, and multiplex immunofluorescence staining were obtained from GBM patients in The Preston Robert Tisch Brain Tumor Center BioRepository (accredited by the College of American Pathologists) with approval from the Institutional Review Board. Tissue sections were reviewed by board-certified neuropathologists to confirm histopathological diagnosis of GBM. Samples with >60% tumor cellularity by hematoxylin and eosin (H&E) staining were selected.

Methylation arrays and datasets: DNA/RNA were co-extracted from snap-frozen tumor tissue or cells using Qiagen AllPrep DNA/RNA/miRNA Universal Kit mini kit (Qiagen) or Quick-DNA/RNA™Miniprep Kit respectively, following the manufacturer's protocols. Methylation assays were performed on DNA while quantitative expression analyses were performed on RNA (see below). DNA was provided to the Duke Molecular Genomics Core who performed bisulfite conversion using the Zymo EZ methylation kit followed by Illumina MethylationEPIC Array screening according to the manufacturer's protocols. Arrays were read on an Illumina iScan instrument and data processed using the Minfi package in R to calculate methylation beta values, where beta=0 repressed a completely unmethylated CpG site and beta=1 represents a fully methylated site.

For our GBM patient sample analyses, we used two sets of methylation array data. In the first dataset we used Illumina EPIC array data on 24 extracted samples from Duke GBM patients as described above. The second methylation dataset included paired sets of pre- and posttreatment samples from 21 GBM patients obtained from the Royal Melbourne Hospital (RMH) and University of Melbourne, Department of Surgery Brain Tumor Bank as previously described (Muscat et al., 2018). We used a total of 40 samples from these 21 patients. Briefly, in this second dataset, DNA was extracted from Formalin-fixed paraffin-embedded (FFPE) tumor samples using ReliaPrep™ FFPE gDNA Miniprep System (Promega), and bisulfiite converted using MethyEasy™ Bisulphite Kit (Genetic Signatures) by The Centre for Applied Genomics, The Hospital for Sick Children (Toronto, Canada) per manufacturer's instructions. The Illumina 450K bead array was then performed, and methylation beta values calculated. These paired methylation data were previously deposited in the Gene Expression Omnibus (GEO) under accession number GSE85087. Beta values from TCGA were downloaded using Wanderer (maplab.imppc.org/wanderer/) (Diez-Villanueva et al., 2015) or the Shiny Methylation Analysis Resource Tool (www.bioinfo-zs.com/smartapp/) (Li et al., 2019). For our pan-cancer and tissue-of-origin normal tissue methylation analysis (Figure S1F), both TCGA and datasets downloaded from GEO were used. Methylation datasets from TCGA were used for the pan-cancer solid tumor analysis for both cancer and tissue-of-origin data when available. Normal samples were not available for adrenal cortical carcinoma (ACC), so 6 normal samples from (Legendre et al., 2016) (GSE77871) were used instead. For tissue-of-origin data for both cutaneous (SKCM) and uveal (UVM) melanoma, adjacent normal samples from the TCGASKCM dataset were used. For tissue-of-origin data for all TCGA kidney cancers, a weighted average of the adjacent normal samples from clear cell (KIRC) and papillary cell (KIRP) datasets were used. Methylation datasets for patients with cancers not in TCGA and normal brain samples are summarized as follows:

| | | GEO accession | |
|---|---|---|---|
| | Dataset | number | Reference |
| Normal tissues | Normal fetal brain | GSE69502 | (Price et al., 2016) |
| | | GSE90871 | (Chatterton et al., 2017) |
| | Normal adult brain | GSE66351 | (Gasparoni et al., 2018) |
| | Normal adrenal cortex | GSE77871 | (Legendre et al., 2016) |
| Other primary | ATRT | GSE141363 | (Ho et al., 2020) |
| brain tumors | Medulloblastoma | GSE75153 | Not available |
| | DIPG | GSE50022 | (Buczkowicz et al., 2014) |
| | Pilocytic astrocytoma | GSE77241 | (Jeyapalan et al., 2016) |
| | Ependymoma | GSE104210 | (Pajtler et al., 2018) |
| | PCNSL | GSE92676 | (Nakamura et al., 2017) |
| | Meningioma | GSE83933 | (Agnihotri et al., 2017) |
| Extracranial | SINET | GSE73832 | (Karpathakis et al., 2016) |
| tumors of | Neuroblastoma | GSE120650 | (Ackermann et al., 2018) |
| neuroectoderm | Pheochromocytoma | GSE43293 | (Letouzé et al., 2013) |
| origin | PanNET | GSE117852 | (Chan et al. 2018) |
| | Pituitary adenoma | GSE147548 | (Salomon et al., 2018) |

cg16983159 site-specific methylation qPCR assay: A site-specific qPCR assay for measuring cg16983159 methylation was developed using the method described by Yu et al. (Yu et al., 2019). DNA/RNA were co-extracted from cells using AllPrep DNA/RNA/miRNA Universal Kit mini kit (Qiagen) for decitabine experiments, and Quick-DNA/RNA™Miniprep Kit (manufacturer) for baseline cell expression/methylation, following the manufacturer's protocols. Bisulfite conversion of 25 ng-1 μg gDNA was done using the EpiTect Bisulfite Kit (Qiagen) following the manufacturer's protocol. STING methylation qPCR was performed on 1-10 ng bisulfite converted DNA with TMEM173 forward 5′-TAGAGGAATGGGGGTTTGGT-3′(SEQ ID NO:1) and reverse 5′-TCCTACCTAATATCATCCCCACAA-3′(SEQ ID NO:2) primers (IDT), methylated FAM-TGTAGGAAATGGTTAcGTT-MGB-NFQ (SEQ ID NO:3) and unmethylated VIC-TGTAGGAAATGGTTAtGTTT-MGB-NFQ (SEQ ID NO:4) TaqMan™ MGB probes (ThermoFisher), and HotStar Taq kit (Qiagen). Percent methylation was calculated as 100%×1/1+2ΔCt where ΔCt=FAM−VIC.

Semi-quantitative methylation sequencing assay: Bisulfite treated DNA was PCR amplified for 40 cycles at 62° C. annealing temperature. Amplification reaction—10 μM of each primer, 25 mM MgCl2, 2.5 mM dNTPs, lx buffer, 0.7U HotStarTaq (Qiagen). Amplicons were sent to Azenta for purification and sanger sequencing with difficult template. Sequence traces were aligned. Percent methylation at the CpGs in the amplicons were estimated by comparing the C peak to the T peak.

| Primer (IDT) | Sequence | |
|---|---|---|
| Sting Sanger | GGTTTGAGTTTTGGGGTTAGGG | SEQ ID NO: 5 |
| For-5 | | |
| Sting Sanger | CTACCTTTAACTAAATATATAA | SEQ ID NO: 6 |
| Rev-5 | AATCCTACTC | |
| Sting Sanger | GTTATTTTTAAATTAAGGGTGT | SEQ ID NO: 7 |
| For-6 | TTAGAA | |
| Sting Sanger | CCTCCCTCCTAAATCACACTTT | SEQ ID NO: 8 |
| Rev-6 | TC | |
| TSS seq 2 | TGTGATTTAGGAGGGAGGAGTG | SEQ ID NO: 9 |
| | A | |
| SITNG qPCR | TCCTACCTAATATCATCCCCAC | SEQ ID NO: 10 |
| Rev | AA | |
| Sting Sanger | TTGTGGGGATGATATTAGGTAG | SEQ ID NO: 11 |
| For-8 | GAG | |
| Sting Sanger | ACCTCAACCTCCCAAATAACTA | SEQ ID NO: 12 |
| Rev-8 | AAATTAC | |

Quantitative gene expression analysis: RNA/DNA were co-extracted from cells using Qiagen AllPrep DNA/RNA/miRNA Universal Kit mini kit (Qiagen) for decitabine experiments, and Quick-DNA/RNA™Miniprep Kit for baseline cell expression/methylation, following the manufacturer's protocols. RNA expression was measured by converting 250 ng to 1 μg RNA to 25 cDNA with the iScript cDNA conversion kit (BioRad). Realtime PCR was performed in triplicate on 2-10 ng/L cDNA with the SsoAdvanced Universal SYBR Green Supermix (BioRad) and PrimePCR Primers targeting B-actin (BioRad #qHsa CED0036269) and TMEM173 (BioRad #qHsa CID00010565). Testing was done on 2 biological replicates. Fold change (2^{-ΔΔCt}) was calculated using B-actin as the housekeeping gene.

10× Genomics scRNA-seq library preparation and Illumina sequencing: Three newly diagnosed GBM tumors were selected for scRNA analysis (total 13,482 cells). Tumor samples were enzymatically dissociated using Collagenase A (Sigma), and the single-cell suspension was stained with live-dead (BioLegend) stain to exclude dead cells. The cell suspension was next treated with Fc receptor block followed by CD45 antibody. Stained cells were subjected to a CD45+ and CD45− sorting. Post-sorting enriched tumor and were pooled and processed for droplet-based RNA sequencing following the manufacturer's protocol (10× Genomics). cDNA isolation and gene expression library preparation were completed as per the manufacturer's protocol using Chromium Single Cell 3′ Reagent Kit (10× Genomics). The gene expression libraries were sequenced on the Illumina NovoSeq platform with 150 bp paired-end read configuration.

scRNA-seq data processing, quality control, and cluster annotation: Raw FASTQ files were analyzed using Cell Ranger 3.1 software. Reads were mapped to the prebuilt GRCh38-3.0.0 reference from 10×'s website. Read lengths were hard-trimmed to 10×'s recommendations for 3′ v3 chemistry. Seurat version 3 was used for downstream analysis. The output of cellranger count from each sample was used as separate inputs into a workflow based on the Seurat data analysis package. First, doublets were detected and removed using doubletCells, part of the scran package from the open-source Bioconductor project (Huber et al., 2015). Next, outliers were calculated based on the following three metrics: mitochondrial percentage, number of features, and number of UMI. Cells identified as outliers were excluded. Then, the Seurat method SCTransform was applied. Variance due to mitochondrial content, number of features, number of genes, and the difference between the G2M and S phase scores was regressed out. Next, PCA was performed on the scaled data. Using the first 25 PCA dimensions, FindNeighbors and FindClusters were performed to identify clusters. Clusters were visualized in a Uniform Manifold Approximation and Projection embedding (UMAP) plot. Significantly expressed genes in each cluster compared to the rest of the dataset were identified using the FindAllMarkers method from Seurat. Expression of TMEM173, PTPRC (immune cells), COL3A1 (endothelial cells), and SOX2 (tumor cells) genes were visualized by feature plots.

Multiplex immunofluorescence staining: Serial FFPE sections (5-micron thickness) were stained with CD64 (NBP2-45625, Novus Biologicals), CD3 (85061, Cell Signaling Technology), CD31 (3528, Cell Signaling Technology), Vimentin (M0725, Dako), STING (13647, Cell Signaling Technology), or corresponding mouse (Cell Signaling Technology) or rabbit isotype control (Cell Signaling Technology) antibodies, and the nuclei were subsequently stained with 4,6-diamidino-2-phenylindole solution (PerkinElmer) using automated IHC techniques on Bond-RXm Processing Module (Leica Microsystems), utilizing the Bond Research Detection kit (DS9455, Leica Microsystems) and Opal fluorophores Opal 520Opal 570, Opal 620, Opal 650, Opal 690 (Akoya Biosciences). The stained sections were cover-slipped using Vectashield HardSet Antifade mounting media (H-1400-10, Vector Laboratories). The slides were scanned using the Vectra 3.0 System (Akoya Biosciences); image analysis was performed using the InForm image analysis software (Akoya Biosciences).

Protein Isolation and Western Blotting: Cells were washed 2× with ice cold 1×PBS and lysed on ice with RIPA lysis buffer, containing protease/phosphatase inhibitors, and benzonase. Lysates were spun down for 15 min at 14,000×g at 4° C. Protein was denatured at 95° C. for 5 min, ran on 4-12% NuPage Bis-Tris gels in 1×MOPS buffer, and transferred to PVDF membrane. The membrane was blocked in 5% Blotting grade blocker (BioRad) dissolved in 1×TBS 0.1% TWEEN20. Primary antibody was added according to manufacturer's protocol and incubated overnight at 4° C. Membranes were incubated with secondary HRP antibody (Cell Signaling) and screened with Supersignal Pico ECL (ThermoFisher). A ChemiDoc MP (BioRad) and an iBright 1500FL imager (ThermoFisher) were, used to detect the ECL signal. Primary antibodies were used to the following targets: pSTING (Ser366), STING, cGAS, pTBK1 (Ser172), TBK1, pIRF3 (Ser396), IRF3, pSTAT1 (Tyr701), STAT1, IFIT1, ISG15, DNMT1, β-actin (Cell Signaling), IRF3 (Biolegend).

GBM slice culture assay: Surgically resected, de-identified newly diagnosed GBM tissue was acquired under a Duke IRB approved protocol within hours of resection. Slice culture studies in this manuscript are extended/additional analyses of slice culture data sets reported in a prior study where the pattern of PRR signaling vs virotherapy was compared (Brown et al., 2021). A portion (˜25%) of fresh tissue was analyzed by flow cytometry to determine relative immune cell subset densities using the following flow cytometry panel after 1:20 Human Tru-Stain FcX treatment (BioLegend): CD45-BUV395 (BD Biosciences), CD14-BV421, CD33-BV510, CD49d-BV605, HLA-DR-BV786, CD31-FITC, CD3/19-BUV737, CD11b-APC, CD16-BV711, CD15-APC-fire7, and either CD155-PE or isotype control-PE antibody (all BioLegend); viability stain (7-AAD, BioLegend) was added after staining. Fresh remaining tissue was sliced into quarters, followed by slicing to −2 mm diameter fragments with a #10 scalpel (Hill-Rom). Slices were cultured in RPMI-1640 (Invitrogen) supplemented with 10% FBS (Sigma-Aldrich), 100 ng/ml EGF (R&D Biosystems); 5 μg/ml insulin (Gibco); 100 ng/ml hydrocortisone (Sigma-Aldrich). Samples were treated with mock (endotoxin free water) or 2′3′-cGAMP (5 μg/ml, Invivogen) for 48 h. Supernatant cytokines were measured using the Human Antiviral Legendplex kit and Human Pro-inflammatory Chemokine kit (both BioLegend), per manufacturer's instructions. For analysis of IFIT1 expression single cells suspensions were generated (100 μg/ml Liberase-™ (Sigma-Aldrich) and 10 μg/ml DNAse I (Roche) for 20 min at 37° C. with agitation) from post-treatment tissue slices, stained with Zombie-Aqua viability dye (BioLegend), and fixed/permeabilized using a fixation/permeabilization buffer set kit (Thermo-Fisher), Fc-blocked (1 h), followed by staining with IFIT1 (Cell Signaling Technology) or isotype control (rabbit IgG; Santa Cruz Biotech) overnight. The following day, cells were washed 2× with permeabilization buffer, stained with donkey anti-rabbit IgG-Alexa Fluor 594 and goat antimouse IgG APC (both Biolegend) for 1 h, followed by washing 2× in permeabilization buffer, and staining for antibodies recognizing: CD45-BUV395 (BD Biosciences), CD14-BV421, CD31-FITC, CD11b-Alexafluor 488, and CD3/CD19-BUV737.

For separation of CD14+ cells from GBM single cell suspensions, single cell suspensions were generated as described above, and the human CD14+ selection kit II (STEMCELL Technologies) was used per manufacturer's instructions. One-third of the sample was retained as pre-depletion sample, the remaining material was processed for CD14+ cell isolation. Pre-depleted, CD14+ enriched cells; and CD14+ depleted cells were cell count-normalized between pre- and postdepleted suspensions using a Countess II cell counter with trypan blue viability stain (Thermo-Fisher; CD14 cell density was not adjusted). Samples were plated in 24-well plates and treated with mock or 2′3′-cGAMP. Depletion of CD14 cells was confirmed by flow cytometry using antibodies against CD45-BUV395, CD14-BV421, and CD11b-APC.

Cell treatment with innate agonists: ISD-Naked, ISD-Control, and 2′3′-cGAMP (Invivogen) were dissolved in sterile water according to the manufacturer's protocol. Cell line gDNA was harvested using the Wizard® Genomic DNA Purification Kit, with inclusion of the RNAse treatment step, according to manufacturer's protocol. Cells were seeded at 1.7-2.0×10₅ cells/well in 6-well plates. Agonist treatment was added directly to the media 24 h after cell seeding. Total protein was harvested 4 h and 24 h after agonist treatment.

Cell treatment with decitabine: Decitabine (Millipore-Sigma) was dissolved in DMSO to stock concentration of 10 mM and diluted directly before each treatment in 1×PBS. Cells were seeded at 3×10⁴ cells/well in 6-well plates (protein) or 1.7×10⁵ cells in 10 cm dishes (RNA/DNA). Matched concentrations of DMSO was used as a control. Media was refreshed with treatment daily for 5 days. Cells were harvested for protein, RNA and DNA on day 6, or treated with 2′3′-cGAMP for an additional 4 and 24 h and harvested for protein.

### REFERENCES

Brown, M. C., Mosaheb, M. M., Mohme, M., McKay, Z. P., Holl, E. K., Kastan, J. P., Yang, Y., Beasley, G. M., Hwang, E. S., Ashley, D. M., et al. (2021). Viral infection of cells within the tumor microenvironment mediates antitumor immunotherapy via selective TBK1-IRF3 signaling. Nature Communications 12, 1858.
Gasparoni, G., Bultmann, S., Lutsik, P., Kraus, T. F. J., Sordon, S., Vlcek, J., Dietinger, V., Steinmaurer, M., Haider, M., Mulholland, C. B., et al. (2018). DNA methylation analysis on purified neurons and glia dissects age and Alzheimer's disease-specific changes in the human cortex. Epigenetics & Chromatin 11, 41.
Kim, H., Nguyen, N.-P., Turner, K., Wu, S., Gujar, A. D., Luebeck, J., Liu, J., Deshpande, V., Rajkumar, U., Namburi, S., et al. (2020). Extrachromosomal DNA is associated with oncogene amplification and poor outcome across multiple cancers. Nat Genet 52, 891-897.
Kitajima, S., Ivanova, E., Guo, S., Yoshida, R., Campisi, M., Sundararaman, S. K., Tange, S., Mitsuishi, Y., Thai, T. C., Masuda, S., et al. (2019). Suppression of STING Associated with LKB1 Loss in KRAS-Driven Lung Cancer. Cancer Discov 9, 34-45.
Konno, H., Yamauchi, S., Berglund, A., Putney, R. M., Mulé, J. J., and Barber, G. N. (2018). Suppression of STING signaling through epigenetic silencing and missense mutation impedes DNA damage mediated cytokine production. Oncogene 37, 2037-2051.
Wang, Y.-Y., Jin, R., Zhou, G.-P., and Xu, H.-G. (2016). Mechanisms of transcriptional activation of the stimulator of interferon genes by transcription factors CREB and c-Myc. Oncotarget 7, 85049-85057.
Xia, T., Konno, H., Ahn, J., and Barber, G. N. (2016a). Deregulation of STING Signaling in Colorectal Carcinoma Constrains DNA Damage Responses and Correlates With Tumorigenesis. Cell Rep 14, 282-297.
Xia, T., Konno, H., and Barber, G. N. (2016b). Recurrent Loss of STING Signaling in Melanoma Correlates with Susceptibility to Viral Oncolysis. Cancer Res 76, 6747-6759.
Ackermann, S., Cartolano, M., Hero, B., Welte, A., Kahlert, Y., Roderwieser, A., Bartenhagen, C., Walter, E., Gecht, J., Kerschke, L., et al. (2018). A mechanistic classification of clinical phenotypes in neuroblastoma. Science 362, 1165-1170.
Agnihotri, S., Suppiah, S., Tonge, P. D., Jalali, S., Danesh, A., Bruce, J. P., Mamatjan, Y., Klironomos, G., Gonen, L., Au, K., et al. (2017). Therapeutic radiation for childhood cancer drives structural aberrations of NF2 in meningiomas. Nat Commun 8, 186.
Brown, M. C., Mosaheb, M. M., Mohme, M., McKay, Z. P., Holl, E. K., Kastan, J. P., Yang, Y., Beasley, G. M., Hwang, E. S., Ashley, D. M., et al. (2021). Viral infection of cells within the tumor microenvironment mediates antitumor immunotherapy via selective TBK1-IRF3 signaling. Nature Communications 12, 1858.
Buczkowicz, P., Hoeman, C., Rakopoulos, P., Pajovic, S., Letourneau, L., Dzamba, M., Morrison, A., Lewis, P., Bouffet, E., Bartels, U., et al. (2014). Genomic analysis of diffuse intrinsic pontine gliomas identifies three molecular subgroups and recurrent activating ACVR1 mutations. Nat Genet 46, 451-456.
Chan, C. S., Laddha, S. V., Lewis, P. W., Koletsky, M. S., Robzyk, K., Da Silva, E., Torres, P. J., Untch, B. R., Li, J., Bose, P., et al. (2018). ATRX, DAXX or MEN1 mutant pancreatic neuroendocrine tumors are a distinct alpha-cell signature subgroup. Nat Commun 9, 4158.
Chatterton, Z., Hartley, B. J., Seok, M.-H., Mendelev, N., Chen, S., Milekic, M., Rosoklija, G., Stankov, A., Trencevsja-Ivanovska, I., Brennand, K., et al. (2017). In utero exposure to maternal smoking is associated with DNA methylation alterations and reduced neuronal content in the developing fetal brain. Epigenetics Chromatin 10, 4.
Diez-Villanueva, A., Mallona, I., and Peinado, M. A. (2015). Wanderer, an interactive viewer to explore DNA methylation and gene expression data in human cancer. Epigenetics Chromatin 8, 22.
Gasparoni, G., Bultmann, S., Lutsik, P., Kraus, T. F. J., Sordon, S., Vlcek, J., Dietinger, V., Steinmaurer, M., Haider, M., Mulholland, C. B., et al. (2018). DNA methylation analysis on purified neurons and glia dissects age and Alzheimer's disease-specific changes in the human cortex. Epigenetics & Chromatin 11, 41.
Ho, B., Johann, P. D., Grabovska, Y., De Dieu Andrianteranagna, M. J., Yao, F., Fruhwald, M., Hasselblatt, M., Bourdeaut, F., Williamson, D., Huang, A., et al. (2020). Molecular subgrouping of atypical teratoid/rhabdoid tumors-a reinvestigation and current consensus. Neuro Oncol 22, 613-624.
Huber, W., Carey, V. J., Gentleman, R., Anders, S., Carlson, M., Carvalho, B. S., Bravo, H. C., Davis, S., Gatto, L., Girke, T., et al. (2015). Orchestrating high-throughput genomic analysis with Bioconductor. Nat Methods 12, 115-121.
Jeyapalan, J. N., Doctor, G. T., Jones, T. A., Alberman, S. N., Tep, A., Haria, C. M., Schwalbe, E. C., Morley, I. C. F., Hill, A. A., LeCain, M., et al. (2016). DNA methylation analysis of paediatric lowgrade astrocytomas identifies a tumour-specific hypomethylation signature in pilocytic astrocytomas. Acta Neuropathol Commun 4, 54.
Karpathakis, A., Dibra, H., Pipinikas, C., Feber, A., Morris, T., Francis, J., Oukrif, D., Mandair, D., Pericleous, M., Mohmaduvesh, M., et al. (2016). Prognostic Impact of Novel Molecular Subtypes of Small Intestinal Neuroendocrine Tumor. Clin Cancer Res 22, 250-258.
Legendre, C. R., Demeure, M. J., Whitsett, T. G., Gooden, G. C., Bussey, K. J., Jung, S., Waibhav, T., Kim, S., and Salhia, B. (2016). Pathway Implications of Aberrant Global Methylation in Adrenocortical Cancer. PLoS One 11, e0150629.
Letouze, E., Martinelli, C., Loriot, C., Burnichon, N., Abermil, N., Ottolenghi, C., Janin, M., Menara, M., Nguyen, A. T., Benit, P., et al. (2013). SDH Mutations Establish a Hypermethylator Phenotype in Paraganglioma. Cancer Cell 23, 739-752.
Li, Y., Ge, D., and Lu, C. (2019). The SMART App: an interactive web application for comprehensive DNA methylation analysis and visualization. Epigenetics & Chromatin 12, 71.
Muscat, A. M., Wong, N. C., Drummond, K. J., Algar, E. M., Khasraw, M., Verhaak, R., Field, K., Rosenthal, M. A., and Ashley, D. M. (2018). The evolutionary pattern of mutations in glioblastoma reveals therapy-mediated selection. Oncotarget 9, 7844-7858.
Nakamura, T., Yamashita, S., Fukumura, K., Nakabayashi, J., Tanaka, K., Tamura, K., Tateishi, K., Kinoshita, M., Fukushima, S., Takami, H., et al. (2017). Genome-wide DNA methylation profiling identifies primary central nervous system lymphoma as a distinct entity different from systemic diffuse large B-cell lymphoma. Acta Neuropathol 133, 321-324.
Pajtler, K. W., Wen, J., Sill, M., Lin, T., Orisme, W., Tang, B., Hubner, J.-M., Ramaswamy, V., Jia, S., Dalton, J. D., et al. (2018). Molecular heterogeneity and CXorf67 alterations in posterior fossa group A (PFA) ependymomas. Acta Neuropathol 136, 211-226.
Price, E. M., Penaherrera, M. S., Portales-Casamar, E., Pavlidis, P., Van Allen, M. I., McFadden, D. E., and Robinson, W. P. (2016). Profiling placental and fetal DNA methylation in human neural tube defects. Epigenetics Chromatin 9, 6.
Salomon, M. P., Wang, X., Marzese, D. M., Hsu, S. C., Nelson, N., Zhang, X., Matsuba, C., Takasumi, Y., Ballesteros-Merino, C., Fox, B. A., et al. (2018). The Epigenomic Landscape of Pituitary Adenomas Reveals Specific Alterations and Differentiates Among Acromegaly, Cushing's Disease and Endocrine-Inactive Subtypes. Clin Cancer Res 24, 4126-4136.
Yu, H., Bai, L., Tang, G., Wang, X., Huang, M., Cao, G., Wang, J., and Luo, Y. (2019). Novel Assay for Quantitative Analysis of DNA Methylation at Single-Base Resolution. Clinical Chemistry 65, 664-673.

1. A method of treating a cancer in a subject, the method comprising:
obtaining a sample from the subject comprising a tumor cell,
determining a methylation status of at least one CpG in a TMEM173 (STING) promoter in the sample, and
administering to the subject an appropriate anti-cancer therapeutic agent based on the methylation status determined; wherein the cancer is a cranial cancer or an extracranial cancer of neural crest or neuroectodermal embryologic origin.

2. The method of claim 1, wherein a demethylating agent is administered if one or more CpG is methylated.

3. The method of claim 2, wherein the demethylating agent is decitabine.

4. The method of claim 2, wherein the method further comprises administering an immunotherapy to the subject.

5. (canceled)

6. The method of claim 4, wherein an immunotherapy is administered if one or more CpG is not methylated, wherein the immunotherapy comprises at least one of an immune checkpoint inhibitor, a vaccine, immunologic adjuvant or oncolytic virus.

7. (canceled)

8. The method of claim 1, wherein the methylation status of at least one CpG in the STING promoter is selected from the CpG sites corresponding to the nucleotides at positions 39, 51, 144, 157, 233, 251 or 279 of SEQ ID NO: 15 or the CpG at cg16983159.

9. (canceled)

10. The method of claim 1, wherein the cancer is a cranial cancer selected from a brain cancer, glioma, glioblastoma (GBM), medulloblastoma, and pituitary adenoma or wherein the cancer is an extracranial cancer of neuroectodermal embryologic origin selected from a neuroblastoma, pancreatic neuroendocrine cancer, medulloblastoma, pituitary adenoma and pheochromocytoma.

11. (canceled)

12. The method of claim 1, wherein the sample is selected from the group consisting of tissues, cells, biopsies, resected tumor sections, blood, lymph, serum, plasma, urine, saliva, mucus and tears.

13. The method of claim 1, wherein the method further comprises administering at least one additional therapeutic agent to the subject.

14. The method of claim 1, wherein the method further comprises resecting one or more tumors from the subject prior to collecting the sample, while collecting the sample, after collecting the sample or after administering the anti-cancer therapeutic agent.

15. A method of predicting responsiveness of a cancer in a subject to an immunotherapy, the method comprising:
obtaining a sample from the subject comprising a tumor cell,
determining a methylation status of at least one CpG in a TMEM173 (STING) promoter in the sample, and
administering an immunotherapy to the subject if one or more CpG is unmethylated;
wherein the cancer is a cranial cancer or an extracranial cancer of neural crest or neuroectodermal embryologic origin, a brain cancer, glioma, glioblastoma, medulloblastoma, and pituitary adenoma.

16. (canceled)

17. The method of claim 15, wherein the cancer is an extracranial cancer of neuroectodermal embryologic origin selected from a neuroblastoma, pancreatic neuroendocrine cancer, medulloblastoma, pituitary adenoma and pheochromocytoma.

18. The method of claim 15, wherein the immunotherapy comprises at least one of an immune checkpoint inhibitor, vaccine, immunologic adjuvant, and oncolytic virus.

19. The method of claim 15, wherein the method further comprises administering at least one additional therapeutic agent to the subject.

20. The method of claim 15, wherein the methylation status of at least one CpG in the STING promoter is selected from the CpG sites corresponding to the nucleotides at positions 39, 51, 144, 157, 233, 251 or 279 of SEQ ID NO: 15 or the CpG at cg16983159.

21. (canceled)

22. A method of treating a cancer in a subject, the method comprising administering a therapeutically effective amount of a demethylating agent to a subject in need thereof; wherein the cancer is a cranial cancer or an extracranial cancer of neural crest or neuroectodermal embryologic origin, wherein the cranial cancer is selected from a brain cancer, glioma, glioblastoma, medulloblastoma, and pituitary adenoma and the extracranial cancer of neuroectodermal embryologic origin selected from a neuroblastoma, pancreatic neuroendocrine cancer, medulloblastoma, pituitary adenoma and pheochromocytoma.

23. (canceled)

24. (canceled)

25. The method of claim 22, wherein the method further comprises administering at least one additional therapeutic or immunomodulatory agent. agent to the subject.

26. (canceled)

27. The method of any claim 22, wherein the method further comprises:
obtaining a sample comprising a tumor cell from the subject, and
detecting a methylation status of at least one CpG of a TMEM173 (STING) promoter in the sample.

28. The method of claim 27, wherein the methylation status of at least one CpG in the STING promoter is selected from the CpG sites corresponding to the nucleotides at positions 39, 51, 144, 157, 233, 251 or 279 of SEQ ID NO: 15 or the CpG at cg16983159.

29. (canceled)

30. A method for identifying an agent that modulates the methylation pattern of a STING-silenced tumor comprising: (a) contacting a sample comprising a hypermethylated STING promoter with the agent; and (b) analyzing the methylation pattern of the STING promoter in the sample prior to and after contact with the agent, wherein the hypermethylated STING promoter is hypermethylated at a CpG site corresponding to at least one nucleotide at positions 39, 51, 144, 157, 233, 251 or 279 of SEQ ID NO: 15.

31. (canceled)

32. (canceled)


SEQUENCE LISTING (15 sequences)

SEQ ID NO: 1 - DNA, 20 bp, Artificial Sequence
  Synthetic- TMEM173 Forward
tagaggaatgggggtttggt

SEQ ID NO: 2 - DNA, 24 bp, Artificial Sequence
  Synthetic- TMEM173 Reverse
tcctacctaatatcatccccacaa

SEQ ID NO: 3 - DNA, 19 bp, Artificial Sequence
  Synthetic- Methylated FAM
  modified_base (16)..(16): m5c
tgtaggaaatggttacgtt

SEQ ID NO: 4 - DNA, 20 bp, Artificial Sequence
  Synthetic- Unmethylated VIC
tgtaggaaatggttatgttt

SEQ ID NO: 5 - DNA, 22 bp, Artificial Sequence
  Synthetic- STING Sanger Forward 5
ggtttgagttttggggttaggg

SEQ ID NO: 6 - DNA, 32 bp, Artificial Sequence
  Synthetic- Sting Sanger Reverse 5
ctacctttaactaaatatataaaatcctactc

SEQ ID NO: 7 - DNA, 28 bp, Artificial Sequence
  Synthetic- Sting Sanger Forward 6
gttatttttaaattaagggtgtttagaa

SEQ ID NO: 8 - DNA, 24 bp, Artificial Sequence
  Synthetic- Sting Sanger Reverse 6
cctccctcctaaatcacacttttc

SEQ ID NO: 9 - DNA, 23 bp, Artificial Sequence
  Synthetic- TSS seq 2
tgtgatttaggagggaggagtga

SEQ ID NO: 10 - DNA, 24 bp, Artificial Sequence
  Synthetic- Sting qPCR Reverse
tcctacctaatatcatccccacaa

SEQ ID NO: 11 - DNA, 25 bp, Artificial Sequence
  Synthetic- Sting Sanger Forward 8
ttgtggggatgatattaggtaggag

SEQ ID NO: 12 - DNA, 29 bp, Artificial Sequence
  Synthetic- Sting Sanger Reverse 8
acctcaacctcccaaataactaaaattac

SEQ ID NO: 13 - DNA, 300 bp, Homo sapiens
  misc_feature: Sting promoter region
  modified_base (39)..(39): m5c
  modified_base (51)..(51): m5c
  modified_base (144)..(144): m5c
  modified_base (157)..(157): m5c
  modified_base (233)..(233): m5c
  modified_base (251)..(251): m5c
  modified_base (279)..(279): m5c
tctcctgcctcagcctcccaagtagctaagattacaagcgcccaccaccacgcctggctaatttttgtttttagtagagatggggtttcaccatgttgctcaggctggtcttgaactcctgacctcaagtgatccacccatctcggcctcccaaagcgctgggattacaggcatgagccactgtgccaggcctgcaattacttttgctcctacctaatatcatccccacaaccgccttctgggcagaaaccggcaggctctcttggagaagtcacaggcgtggccatttcctgcaaagag

SEQ ID NO: 14 - DNA, 600 bp, Homo sapiens
tctcctgcctcagcctcccaagtagctaagattacaagcgcccaccaccacgcctggctaatttttgtttttagtagagatggggtttcaccatgttgctcaggctggtcttgaactcctgacctcaagtgatccacccatctcggcctcccaaagcgctgggattacaggcatgagccactgtgccaggcctgcaattacttttgctcctacctaatatcatccccacaaccgccttctgggcagaaaccggcaggctctcttggagaagtcacaggcgtggccatttcctgcaaagagccaaacccccattcctctgtgcccctcctctcccaccaagtgctttataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggtgagtgtttctaa

SEQ ID NO: 15 - DNA, 300 bp, Homo sapiens; identical to SEQ ID NO: 13